(19) United States
(12) Patent Application Publication
(10) Pub. No.: US 2025/0213550 A1
(43) Pub. Date: Jul. 3, 2025

(54) COMPOUNDS AND METHODS FOR TREATING INFLAMMATORY BOWEL DISEASE

(71) Applicant: Gilead Sciences, Inc., Foster City, CA (US)

(72) Inventors: Elijah J. Weber, San Francisco, CA (US); Ahmed A. Othman, Dublin, CA (US); Ingrid J. Chang, Denver, CO (US); Matthew T. McKevitt, Seattle, WA (US)

(21) Appl. No.: 18/974,373

(22) Filed: Dec. 9, 2024

Related U.S. Application Data
(60) Provisional application No. 63/608,750, filed on Dec. 11, 2023.

(51) Int. Cl. A61K 31/4725; A61K 9/20; A61K 31/675; A61P 29/00

(52) U.S. Cl. CPC A61K 31/4725; A61K 9/2009; A61K 9/2027; A61K 9/2054; A61K 31/675; A61P 29/00

(57) ABSTRACT

The present disclosure relates generally to methods of using inhibitors of tumor progression locus 2 (TPL2) kinase for treating, stabilizing, or lessening the severity or progression of inflammatory bowel disease (IBD), including Crohn's disease (CD) and ulcerative colitis (UC), and pharmaceutical compositions including TPL2 kinase inhibitors for use in such methods.

### CROSS REFERENCE TO RELATED APPLICATIONS

This application claims the benefit of U.S. Provisional Application No. 63/608,750, filed on Dec. 11, 2023, the entire contents of which is hereby incorporated by reference in its entirety.

### TECHNICAL FIELD

The present disclosure relates to inhibitors of tumor progression locus 2 (TPL2) kinase, including methods of treating inflammatory bowel disease (JBD) with such inhibitors and pharmaceutical compositions including such inhibitors.

### BACKGROUND

Inflammatory bowel diseases (JBD), including Crohn's disease (CD) and ulcerative colitis (UC), are chronic, relapsing, and remitting diseases thought to be caused by inappropriate inflammatory responses to commensal microbiota in genetically predisposed individuals. There is an increasing incidence in newly industrialized countries and high prevalence (up to 0.5% of the population) in western countries.

Symptoms of UC, a disease isolated to the colon, include rectal bleeding, abdominal pain, increased stool frequency, and urgency. Typically, treatment includes aminosalicylates (mild disease) and immunomodulators, steroids, anti-tumor necrosis factor (TNF) agents, vedolizumab, and surgical resection or colectomy (moderate to severe disease). Symptoms of CD, which usually affects the entire gastrointestinal tract, include chronic diarrhea, abdominal pain, weight loss, anorexia, and fever. Treatment of CD can depend on disease site and severity, and typically includes aminosalicylates (mild disease), immunomodulators (moderate disease), and steroids to induce remission. Steroid-refractory disease is conventionally treated with anti-TNF agents, immunomodulators, vedolizumab, or ustekinumab.

Despite a variety of agents for IBD, response and remission rates remain low, and there accordingly remains a significant unmet need for safe and effective treatments for IBD, including CD and UC.

### SUMMARY

Provided herein is a method of treating inflammatory bowel disease (IBD) in a patient in need thereof, comprising administering to the patient a prodrug of Compound A:

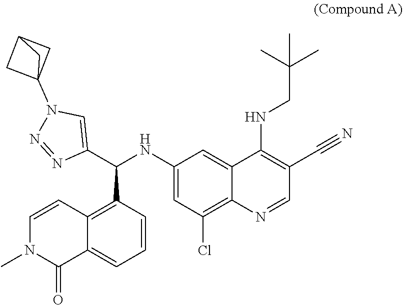 [SMILES: Cn1ccc2c([C@H](Nc3cc(Cl)c4ncc(C#N)c(NCC(C)(C)C)c4c3)c3cn(C45CC(C4)C5)nn3)cccc2c1=O]

or a pharmaceutically acceptable salt of the prodrug, wherein the prodrug, or a pharmaceutically acceptable salt thereof, is administered in a dosage of about 75 mg/dose to about 1,500 mg/dose on a free base basis.

Also provided herein is a method of treating IBD in a patient in need thereof, comprising administering to the patient a prodrug of Compound A, wherein the prodrug is administered in a dosage of about 75 mg/dose to about 1,500 mg/dose.

Also provided is a method of treating IBD in a patient in need thereof, comprising administering to the patient a prodrug which is:

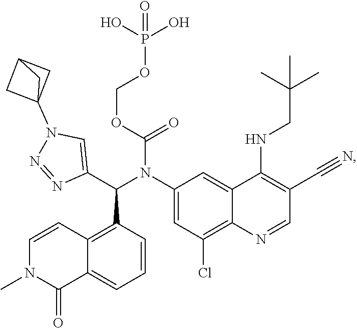 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

wherein the prodrug is administered in a dosage of about 75 mg/dose to about 1,500 mg/dose.

Also provided herein is a method for treating IBD in a patient in need thereof, comprising administering to the patient a therapeutically effective amount of Compound 1:

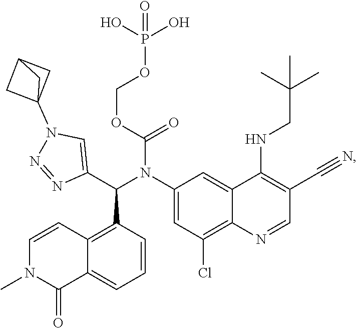 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

or a pharmaceutically acceptable salt thereof, wherein Compound 1 is administered in a dosage of about 75 mg/dose to about 1,500 mg/dose. Also provided herein is a method of treating IBD in a patient in need thereof, comprising administering to the patient a prodrug of Compound A, or a pharmaceutically acceptable salt of the prodrug, wherein the prodrug, or a pharmaceutically acceptable salt thereof, is administered with food.

Also provided herein is a method of treating IBD in a patient in need thereof, comprising administering to the patient a prodrug of Compound A, wherein the prodrug is administered with food.

Also provided is a method of treating IBD in a patient in need thereof, comprising administering to the patient a prodrug which is:

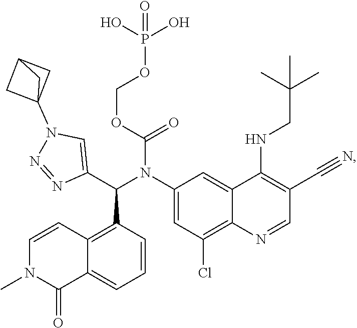 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

wherein the prodrug is administered with food.

Also provided herein is a method of treating IBD in a patient in need thereof, comprising administering to the patient a prodrug of Compound A, or a pharmaceutically acceptable salt of the prodrug, wherein the prodrug, or a pharmaceutically acceptable salt thereof, is administered in a first dosage of about 150 mg/dose to about 1,500 mg/dose on a free base basis, for a first period of up to about 24 weeks; and then in a second dosage for a second period.

Also provided herein is a method of treating IBD in a patient in need thereof, comprising administering to the patient a prodrug of Compound A, wherein the prodrug is administered in a first dosage of about 150 mg/dose to about 1,500 mg/dose, for a first period of up to about 24 weeks; and then in a second dosage for a second period.

Also provided is a method of treating IBD in a patient in need thereof, comprising administering to the patient a prodrug which is:

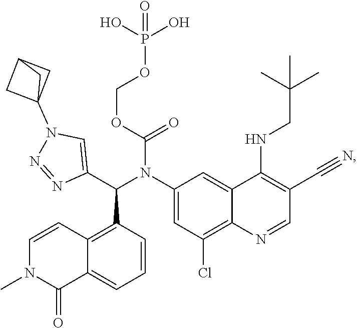 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

wherein the prodrug is administered in a first dosage of about 150 mg/dose to about 1,500 mg/dose, for a first period of up to about 24 weeks; and then in a second dosage for a second period.

Also provided herein is a method for treating IBD in a patient in need thereof, comprising administering to the patient a therapeutically effective amount of a compound of Compound 1:

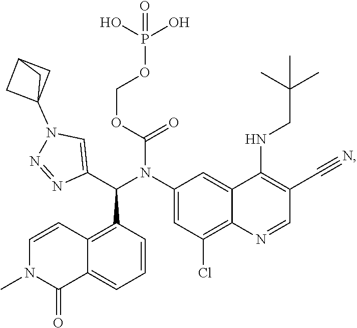 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

or a pharmaceutically acceptable salt thereof, wherein Compound 1 is administered in a first dosage of about 150 mg/dose to about 1,500 mg/dose, for a first period of up to about 24 weeks; and then in a second dosage for a second period.

Also provided herein is a method for treating IBD in a patient in need thereof, comprising administering to the patient a therapeutically effective amount of a compound of Compound 1:

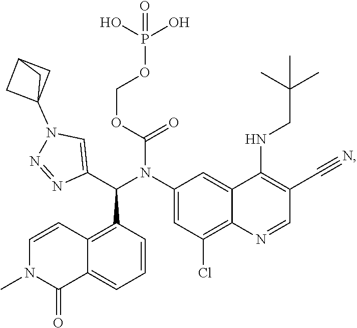 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

or a pharmaceutically acceptable salt thereof, wherein Compound 1 is administered in a first dosage of about 150 mg/dose, or about 300 mg/dose, or about 600 mg/dose, for a first period of up to about 12 weeks; and then in a second dosage of about 150 mg/dose, or about 300 mg/dose, or about 600 mg/dose, for a second period.

Also provided herein is a method for treating IBD in a patient in need thereof, comprising administering to the patient a therapeutically effective amount of a compound of Compound 1:

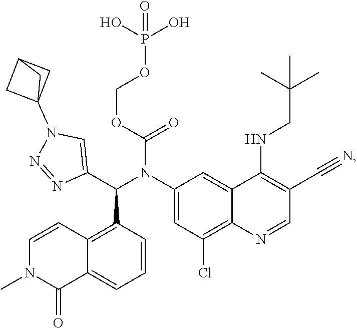 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

or a pharmaceutically acceptable salt thereof, wherein Compound 1 is administered in a first dosage of about 150 mg/dose, or about 300 mg/dose, or about 600 mg/dose, for a first period of up to about 12 weeks; determining whether the patient achieved an adequate response to the first dosage after the first period; and then administering a second dosage of about 150 mg/dose, or about 300 mg/dose, or about 600 mg/dose, for a second period.

Also provided is a pharmaceutical composition comprising a prodrug of Compound A, or a pharmaceutically acceptable salt of the prodrug, wherein the pharmaceutical composition further comprises a diluent, a disintegrant, or a lubricant, or any combination thereof.

Also provided is a pharmaceutical composition comprising a prodrug of Compound A, wherein the pharmaceutical composition further comprises a diluent, a disintegrant, or a lubricant, or any combination thereof.

Also provided is a pharmaceutical composition comprising a prodrug which is:

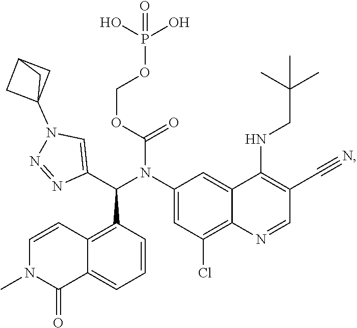 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

wherein the pharmaceutical composition further comprises a diluent, a disintegrant, or a lubricant, or any combination thereof.

Also provided is an oral dosage form comprising a prodrug of Compound A, or a pharmaceutically acceptable salt of the prodrug, and a pharmaceutically acceptable excipient, wherein the oral dosage form is formulated to achieve one or more pharmacokinetic characteristics selected from an arithmetic mean steady-state AUCₜₐᵤ of Compound A of about 10 h*μg/mL to about 80 h*μg/mL; an arithmetic mean steady-state Cₘₐₓ of Compound A of about 0.9 g/mL to about 9 g/mL; a median steady-state Tₘₐₓ of Compound A of about 1.5 hours to about 6 hours; and a median steady-state t_{1/2} of Compound A of about 18 hours to about 30 hours, upon daily oral administration of the dosage form to a human patient in a fasted state.

Also provided is an oral dosage form comprising a prodrug of Compound A, and a pharmaceutically acceptable excipient, wherein the oral dosage form is formulated to achieve one or more pharmacokinetic characteristics selected from an arithmetic mean steady-state AUCₜₐᵤ of Compound A of about 10 h*μg/mL to about 80 h*μg/mL; an arithmetic mean steady-state Cₘₐₓ of Compound A of about 0.9 g/mL to about 9 g/mL; a median steady-state Tₘₐₓ of Compound A of about 1.5 hours to about 6 hours; and a median steady-state t_{1/2} of Compound A of about 18 hours to about 30 hours, upon daily oral administration of the dosage form to a human patient in a fasted state.

### BRIEF DESCRIPTION OF DRAWINGS

FIG. 1 is a median (Q1, Q3) plot for pERK signal percent inhibition by dose cohort and normalized to baseline after single ascending doses of Compound 1.

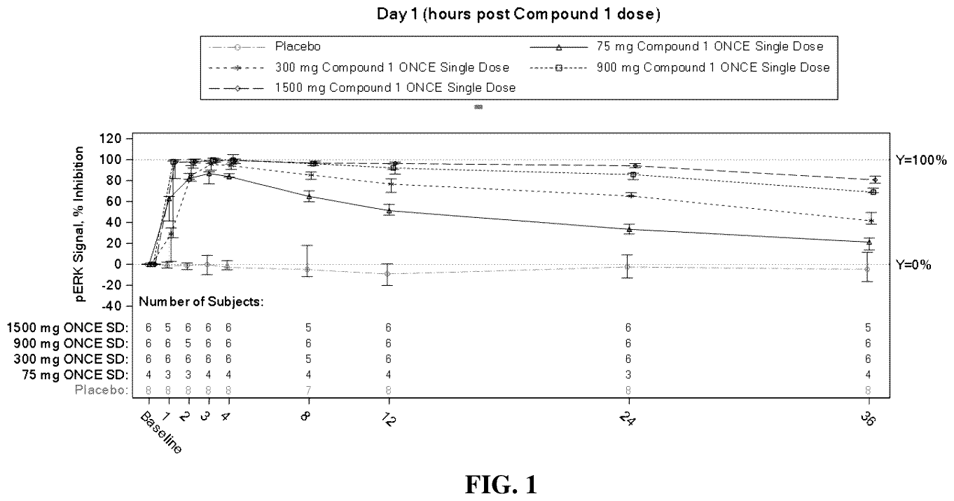

FIG. 2 is a median (Q1, Q3) plot for pERK signal percent inhibition by dose cohort normalized to baseline after multiple ascending doses of Compound 1 and reported one day after first dose.

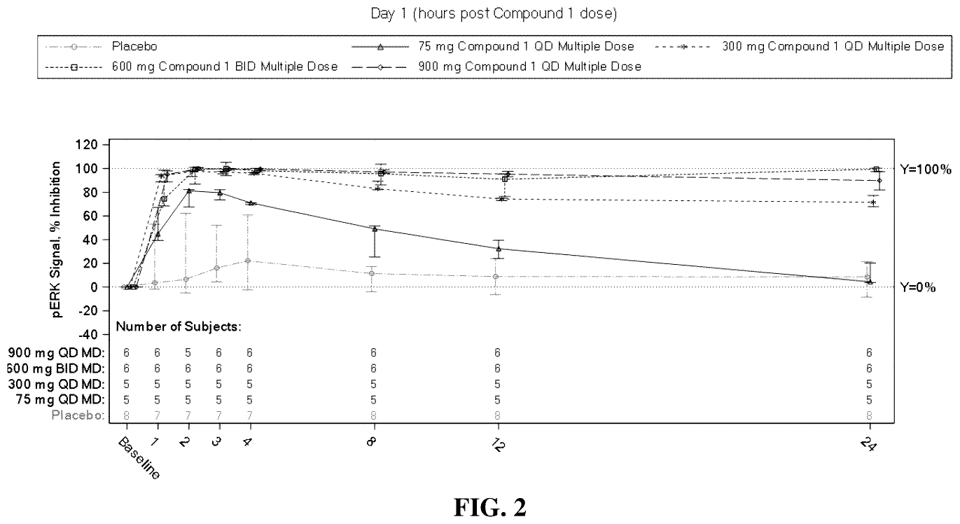

FIG. 3 is a median (Q1, Q3) plot for pERK signal percent inhibition by dose cohort normalized to baseline after multiple ascending doses of Compound 1 and reported 10 days after first dose.

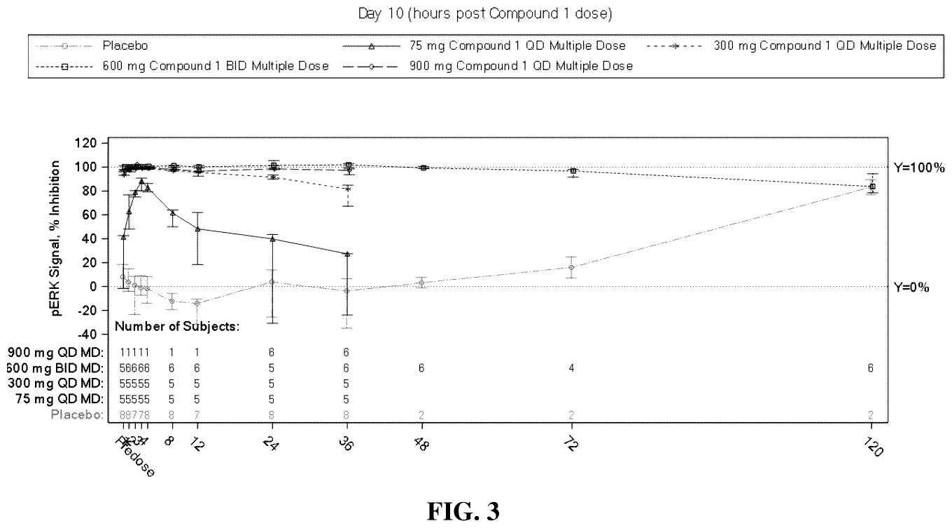

FIG. 4 is simulated steady state PK profiles reflecting Compound A plasma concentration (ng/mL) following 150 mg, 300 mg, and 600 mg doses of Compound 1.

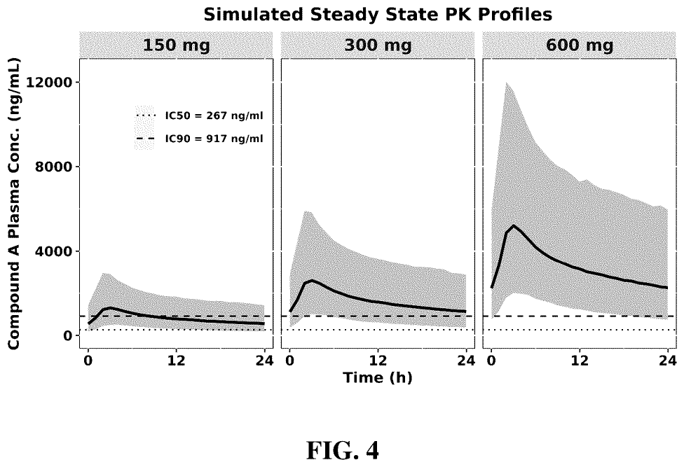

### DETAILED DESCRIPTION

The present disclosure relates to methods for treating inflammatory bowel disease (IBD) with a prodrug of Compound A:

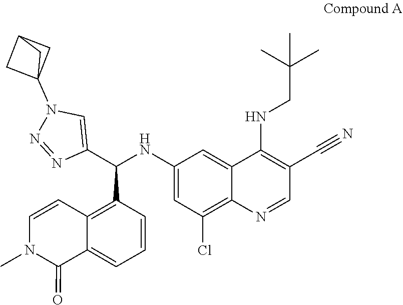 [SMILES: Cn1ccc2c([C@H](Nc3cc(Cl)c4ncc(C#N)c(NCC(C)(C)C)c4c3)c3cn(C45CC(C4)C5)nn3)cccc2c1=O]

or a pharmaceutically acceptable salt of the prodrug, and to pharmaceutical compositions and oral dosage forms including such prodrugs.

Compound A is a potent inhibitor of tumor progression locus 2 (TPL2) kinase, a cytoplasmic serine/threonine kinase and the primary regulator of extracellular signal-regulated kinase (ERK)-mediated gene expression downstream of multiple proinflammatory stimuli. Upon stimulation, the TPL2-ERK pathway is activated in a broad range of immune cells and drives the production of pro-inflammatory cytokines including TNFα, IL-1β, IL-6, and IL-8, all of which are cytokines involved in the pathogenesis of UC. TPL2 RNA is upregulated in ulcerative colitis (UC) patient colonic biopsies, and its expression correlates with IBD-associated genes. A TPL2 gain of function polymorphism has been identified as a risk allele in UC.

The synthesis and characterization of certain prodrugs of Compound A, or pharmaceutically acceptable salts of the prodrugs, and methods of use thereof, are disclosed in U.S. Pat. No. 10,947,259, which is incorporated herein by reference in its entirety for all purposes.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Definitions and General Parameters

The following description sets forth exemplary methods, parameters and the like. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

The terms “treatment” or “treating,” as used herein, refer to an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The terms “prevention” or “preventing,” as used herein, refer to any treatment of a disease or condition that causes the clinical symptoms of the disease or condition not to develop. Compounds may, in some embodiments, be administered to a patient (including a human) who is at risk or has a family history of the disease or condition.

As used herein, the term “patient” refers to any animal including mammals such as mice, rats, other rodents, rabbits, dogs, cats, swine, cattle, sheep, horses, primates, and humans. In some embodiments, the patient is human.

A “pharmaceutical composition” is intended to include the combination of an active agent with a carrier, inert or active, making the composition suitable for diagnostic or therapeutic use in vitro, in vivo, or ex vivo.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. The therapeutically effective amount may vary depending on the subject, and disease or condition being treated, the weight and age of the subject, the severity of the disease or condition, and the manner of administering, which can readily be determined by one or ordinary skill in the art.

The term “prodrug,” as used herein, refers to a biologically inactive derivative of a drug that, upon administration to the patient, can be converted to a parent drug according to some chemical or enzymatic pathway. For example, when a prodrug of the compound of Compound A, or a pharmaceutically acceptable salt thereof, is administered, the prodrug, or the pharmaceutically acceptable salt thereof, can be converted to the compound of Compound A.

The term “converted substantially,” as used herein in reference to a prodrug, refers to conversion of greater than 50% of a prodrug (e.g., a prodrug of the compound of Compound A) to a parent compound (e.g., the compound of Compound A). For example, the term “converted substantially” can refer to conversion of greater than 50%, greater than 60%, greater than 70%, greater than 80%, greater than 90%, greater than 95%, or greater than 99% of the prodrug of the compound of Compound A, or the pharmaceutically acceptable salt thereof, to the compound of Compound A.

The term “steady-state,” as used herein, is reached when the quantity of a drug eliminated in a given unit of time equals the quantity of the drug that reaches systemic circulation in the unit of time. A “steady-state” pharmacokinetic characteristic means the characteristic can be achieved upon reaching steady-state.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

“Haloalkoxy” refers to an alkoxy group as defined above, wherein one or more hydrogen atoms are replaced by a halogen.

“Alkylthio” refers to the group “alkyl-S—”.

“Acyl” refers to a group —C(O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cylcohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently selected from the group consisting of hydrogen, alkyl, aryl, haloalkyl, or heteroaryl; each of which may be optionally substituted.

“Amino” refers to the group —NR^{y}R^{z} wherein R^{y} and R^{z} are independently selected from the group consisting of hydrogen, alkyl, haloalkyl, aryl, or heteroaryl; each of which may be optionally substituted.

“Amidino” refers to —C(NH)(NH₂).

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthylenyl, fluorenyl, and anthracenyl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Azido” refers to —N₃.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently selected from the group consisting of hydrogen, alkyl, aryl, haloalkyl, or heteroaryl; each of which may be optionally substituted.

“Carboxyl” refers to —C(O)OH.

“Carboxyl ester” refers to both —OC(O)R and —C(O)OR, wherein R is hydrogen, alkyl, cycloalkyl, heterocyclyl, aryl, heteroalkyl, or heteroaryl; each of which may be optionally substituted, as defined herein.

“Cyano” or “carbonitrile” refers to the group —CN.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Guanidino” refers to —NHC(NH)(NH₂).

“Hydrazino” refers to —NHNH₂.

“Imino” refers to a group —C(NR)R, wherein each R is alkyl, cycloalkyl, heterocyclyl, aryl, heteroalkyl, or heteroaryl; each of which may be optionally substituted, as defined herein.

“Halogen” or “halo” includes fluoro, chloro, bromo, and iodo. “Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single ring or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. A heterocyclyl may contain one or more oxo and/or thioxo groups. Examples of heterocyclyl groups include pyrrolidinyl, piperidinyl, piperazinyl, oxetanyl, dioxolanyl, azetidinyl, and morpholinyl. As used herein, the term “bridged-heterocyclyl” refers to a four- to ten-membered cyclic moiety connected at two non-adjacent atoms of the heterocyclyl with one or more (e.g., 1 or 2) four- to ten-membered cyclic moiety having at least one heteroatom where each heteroatom is independently selected from nitrogen, oxygen, and sulfur. As used herein, bridged-heterocyclyl includes bicyclic and tricyclic ring systems. Also used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl rings include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 1-oxo-1,2,3,4-tetrahydroisoquinolinyl, 1-oxo-1,2-dihydroisoquinolinyl, 4,5,6,7-tetrahydrothieno[2,3-c]pyridinyl, indolinyl, and isoindolinyl, where the heterocyclyl can be bound via either ring of the fused system.

“Hydroxy” or “hydroxyl” refers to the group —OH. “Hydroxyalkyl” refers to an unbranched or branched alkyl group as defined above, wherein one or more hydrogen atoms are replaced by a hydroxyl.

“Oxo” refers to the group (═O) or (O).

“Nitro” refers to the group —NO₂.

“Sulfonyl” refers to the group —S(O)₂R, where R is alkyl, haloalkyl, heterocyclyl, cycloalkyl, heteroaryl, or aryl. Examples of sulfonyl are methylsulfonyl, ethylsulfonyl, phenylsulfonyl, and toluenesulfonyl.

“Alkylsulfonyl” refers to the group —S(O)₂R, where R is alkyl.

“Alkylsulfinyl” refers to the group —S(O)R, where R is alkyl.

“Thiocyanate”-SCN.

“Thiol” refers to the group —SR, where R is alkyl, haloalkyl, heterocyclyl, cycloalkyl, heteroaryl, or aryl.

“Thioxo” or “thione” refer to the group (═S) or (S).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl, the last-mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorus, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogues” of compounds of Formula I in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, such as a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I.

The concentration of such a heavier isotope, specifically deuterium, may be defined by an isotopic enrichment factor. In the compounds of this disclosure any atom not specifically designated as a certain isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In some cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

### Methods

Provided herein is a method of treating inflammatory bowel disease (IBD) in a patient in need thereof, including administering to the patient a prodrug of Compound A or a pharmaceutically acceptable salt of the prodrug, wherein the prodrug, or a pharmaceutically acceptable salt thereof, is administered in a dosage of about 75 mg/dose to about 1,500 mg/dose on a free base basis. In some embodiments, the method includes administering to the patient a prodrug of Compound A, wherein the prodrug is administered in a dosage of about 75 mg/dose to about 1,500 mg/dose. In some embodiments, the IBD is ulcerative colitis (UC) or Crohn's disease (CD). In some embodiments, the patient in need thereof is a human.

In some embodiments, the prodrug is a compound of Formula I:

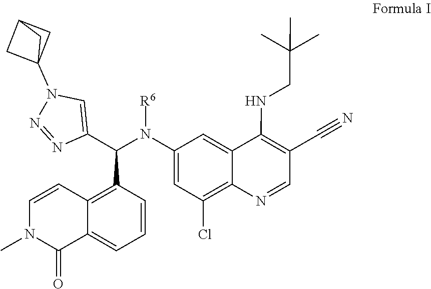

or a pharmaceutically acceptable salt thereof, wherein
R⁶ is —C(O)O—R¹⁶—OP(O)(OR¹²)₂, —C(O)—R¹⁶—OP(O)(OR¹²)₂, —R¹⁶—OP(O)(OR¹²)₂, —C(O)O—R¹⁶—OR¹⁷, —C(O)O—R¹⁶—OH, —C(O)O—R¹⁶—OC(O)R¹⁷, —C(O)—C(O)OR¹², or —C(O)O—R¹⁶—OC(O)R¹⁷NH₂;
each R¹² is independently hydrogen or C₁₋₉ alkyl;
R¹⁶ is —C₁₋₃ alkyl or cyclopropyl, each optionally substituted with one to four C₁₋₃ alkyl or cyclopropyl; and
R¹⁷ is C₁₋₉ alkyl, cycloalkyl, or heterocyclyl, each optionally substituted with one to three R¹⁶.

In certain such embodiments of Formula I, R⁶ is —C(O)O—R¹⁶—OP(O)(OR¹²)₂. In some embodiments, each R¹² is hydrogen. In some embodiments, R¹⁶ is C₁₋₃ alkyl. In some embodiments, R⁶ is

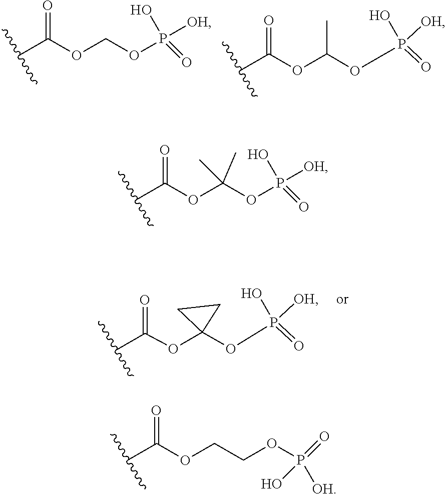 [SMILES: *C(=O)OCOP(=O)(O)O ; *C(=O)OC(C)OP(=O)(O)O ; *C(=O)OC(C)(C)OP(=O)(O)O ; *C(=O)OC1(OP(=O)(O)O)CC1 ; *C(=O)OCCOP(=O)(O)O]

In some embodiments, R⁶ is

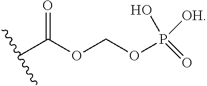 [SMILES: *C(=O)OCOP(=O)(O)O]

In some embodiments, R⁶ is

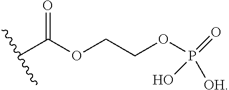 [SMILES: *C(=O)OCCOP(=O)(O)O]

In some embodiments, the prodrug is a compound of Table 1, or a pharmaceutically acceptable salt thereof.

TABLE 1

| | |
|---|---|
| Compound 1 | 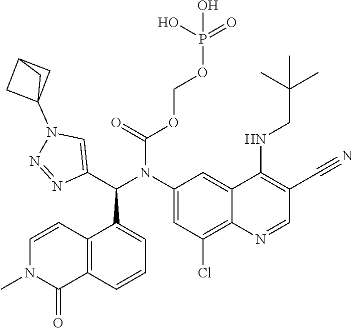 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O] |
| Compound 2 | 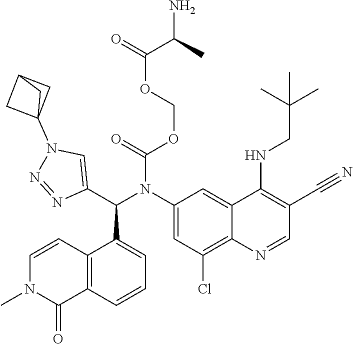 [SMILES: C[C@H](N)C(=O)OCOC(=O)N(c1cc(Cl)c2ncc(C#N)c(NCC(C)(C)C)c2c1)[C@H](c1cn(C23CC(C2)C3)nn1)c1cccc2c(=O)n(C)ccc12] |
| Compound 3 | 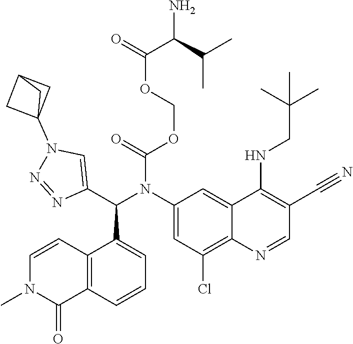 [SMILES: CC(C)[C@H](N)C(=O)OCOC(=O)N(c1cc(Cl)c2ncc(C#N)c(NCC(C)(C)C)c2c1)[C@H](c1cn(C23CC(C2)C3)nn1)c1cccc2c(=O)n(C)ccc12] |
| Compound 4 | 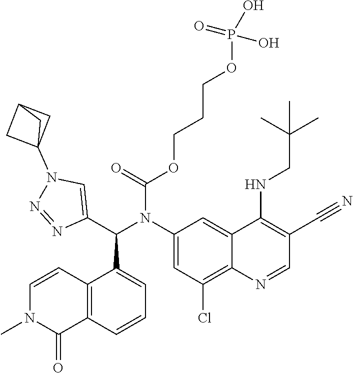 [SMILES: Cn1ccc2c([C@@H](c3cn(C45CC(C4)C5)nn3)N(C(=O)OCCCOP(=O)(O)O)c3cc(Cl)c4ncc(C#N)c(NCC(C)(C)C)c4c3)cccc2c1=O] |
| Compound 5 | 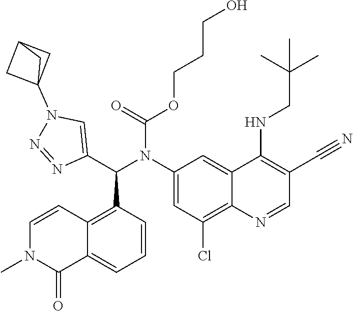 [SMILES: Cn1ccc2c([C@@H](c3cn(C45CC(C4)C5)nn3)N(C(=O)OCCCO)c3cc(Cl)c4ncc(C#N)c(NCC(C)(C)C)c4c3)cccc2c1=O] |
| Compound 17 | 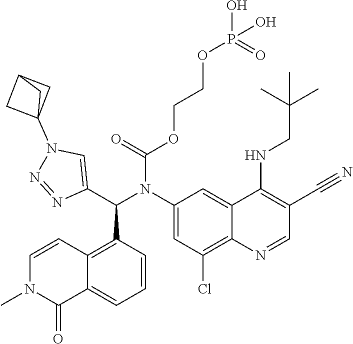 [SMILES: Cn1ccc2c([C@@H](c3cn(C45CC(C4)C5)nn3)N(C(=O)OCCOP(=O)(O)O)c3cc(Cl)c4ncc(C#N)c(NCC(C)(C)C)c4c3)cccc2c1=O] |

In some embodiments, the prodrug is Compound 1. For example, in certain such embodiments, the method comprises administering to the patient a prodrug which is Compound 1, wherein the prodrug is administered in a dosage of about 75 mg/dose to about 1,500 mg/dose.

In some embodiments, the prodrug (e.g., a compound of Formula I, or Compound 1, Compound 2, Compound 3, Compound 4, Compound 5, or Compound 17), or a pharmaceutically acceptable salt thereof, is administered in a dosage of about 75 mg/dose to about 900 mg/dose, about 75 mg/dose to about 600 mg/dose, about 75 mg/dose to about 300 mg/dose, about 150 mg/dose to about 1,500 mg/dose, about 150 mg/dose to about 900 mg/dose, about 150 mg/dose to about 600 mg/dose, about 150 mg/dose to about 300 mg/dose, about 300 mg/dose to about 1,500 mg/dose, about 300 mg/dose to about 900 mg/dose, or about 300 mg/dose to about 600 mg/dose, on a free base basis. In some embodiments, the prodrug, or a pharmaceutically acceptable salt thereof, is administered in a dosage of about 75 mg/dose, 150 mg/dose, about 300 mg/dose, about 600 mg/dose, or about 900 mg/dose, on a free base basis.

In some embodiments, the prodrug (e.g., a compound of Formula I, or Compound 1, Compound 2, Compound 3, Compound 4, Compound 5, or Compound 17), or a pharmaceutically acceptable salt thereof, is administered twice daily. In some embodiments, the prodrug, or a pharmaceutically acceptable salt thereof, is administered once daily. In some embodiments, the prodrug, or a pharmaceutically acceptable salt thereof, is administered in a total daily dosage of about 75 mg to about 1,500 mg, about 75 mg to about 1,200 mg, about 75 mg to about 900 mg, about 75 mg to about 600 mg, about 75 mg to about 300 mg, about 150 mg to about 1,500 mg, about 150 mg to about 1,200 mg, about 150 mg to about 900 mg, about 150 mg to about 600 mg, about 150 mg to about 300 mg, about 300 mg to about 1,500 mg, about 300 mg to about 1,200 mg, about 300 mg to about 900 mg, about 300 mg to about 600 mg, about 600 mg to about 1,500 mg, about 600 mg to about 1,200 mg, or about 600 mg to about 900 mg, on a free base basis. In some embodiments, the prodrug, or a pharmaceutically acceptable salt thereof, is administered in a total daily dosage of about 75 mg, about 150 mg, about 300 mg, about 600 mg, about 900 mg, or about 1,200 mg, on a free base basis.

For example, in some embodiments, the method includes administering Compound 1 to the patient once daily, in a dosage of about 150 mg/dose, about 300 mg/dose, or about 600 mg/dose. In certain such embodiments, the IBD is UC or CD.

In some embodiments, the prodrug, or a pharmaceutically acceptable salt thereof, is administered to a patient in a fasted state. In some embodiments, the prodrug, or a pharmaceutically acceptable salt thereof, is administered to the patient in a fed state. In some embodiments, the prodrug, or a pharmaceutically acceptable salt thereof, is administered with food.

Also provided herein is a method of treating IBD in a patient in need thereof, including administering to the patient a prodrug of Compound A, or a pharmaceutically acceptable salt of the prodrug, wherein the prodrug, or a pharmaceutically acceptable salt thereof, is administered with food. In some embodiments, the method includes administering to the patient a prodrug of Compound A, wherein the prodrug is administered with food. In some embodiments, the IBD is UC or CD.

In some embodiments, the prodrug administered with food is a compound of Formula I, or a pharmaceutically acceptable salt thereof, wherein
R⁶ is —C(O)O—R¹⁶—OP(O)(OR¹²)₂, —C(O)—R¹⁶—OP(O)(OR¹²)₂, —R¹⁶—OP(O)(OR¹²)₂, —C(O)O—R¹⁶—OR¹⁷, —C(O)O—R¹⁶—OH, —C(O)O—R¹⁶—OC(O)R¹⁷, —C(O)—C(O)OR¹², or —C(O)O—R¹⁶—OC(O)R¹⁷NH₂;
each R¹² is independently hydrogen or C₁₋₉ alkyl;
R¹⁶ is —C₁₋₃ alkyl or cyclopropyl, each optionally substituted with one to four C₁₋₃ alkyl or cyclopropyl; and
R¹⁷ is C₁₋₉ alkyl, cycloalkyl, or heterocyclyl, each optionally substituted with one to three R¹⁶.

In certain such embodiments of Formula I, R⁶ is —C(O)O—R¹⁶—OP(O)(OR¹²)₂. In some embodiments, each R¹² is hydrogen. In some embodiments, R¹⁶ is C₁₋₃ alkyl. In some embodiments, R⁶ is

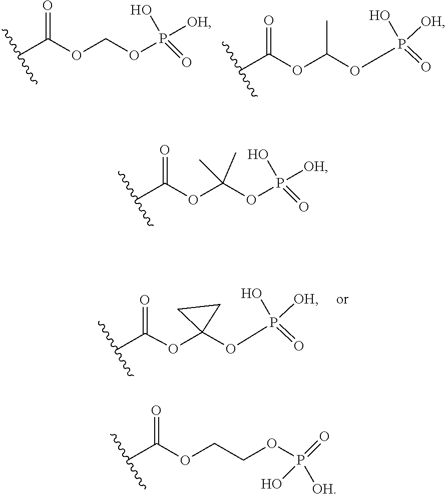 [SMILES: *C(=O)OCOP(=O)(O)O ; *C(=O)OC(C)OP(=O)(O)O ; *C(=O)OC(C)(C)OP(=O)(O)O ; *C(=O)OC1(OP(=O)(O)O)CC1 ; *C(=O)OCCOP(=O)(O)O]

In some embodiments, R⁶ is

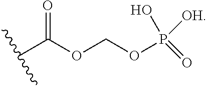 [SMILES: *C(=O)OCOP(=O)(O)O]

In some embodiments, R⁶ is

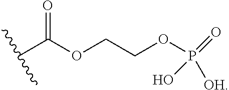 [SMILES: *C(=O)OCCOP(=O)(O)O]

In some embodiments, the prodrug is Compound 1, Compound 2, Compound 3, Compound 4, Compound 5, or Compound 17, or a pharmaceutically acceptable salt thereof. In some embodiments, the prodrug is Compound 1. For example, in certain such embodiments, the method comprises administering to the patient a prodrug which is Compound 1, wherein the prodrug is administered with food.

In some embodiments, the prodrug (e.g., a compound of Formula I, or Compound 1, Compound 2, Compound 3, Compound 4, Compound 5, or Compound 17), or a pharmaceutically acceptable salt thereof, is administered with food, in a dosage of about 75 mg/dose to about 1,500 mg/dose, about 75 mg/dose to about 900 mg/dose, about 75 mg/dose to about 600 mg/dose, about 75 mg/dose to about 300 mg/dose, about 150 mg/dose to about 1,500 mg/dose, about 150 mg/dose to about 900 mg/dose, about 150 mg/dose to about 600 mg/dose, about 150 mg/dose to about 300 mg/dose, about 300 mg/dose to about 1,500 mg/dose, about 300 mg/dose to about 900 mg/dose, or about 300 mg/dose to about 600 mg/dose, on a free base basis. In some embodiments, the prodrug, or a pharmaceutically acceptable salt thereof, is administered with food, in a dosage of about 75 mg/dose, 150 mg/dose, about 300 mg/dose, about 600 mg/dose, or about 900 mg/dose, on a free base basis.

In some embodiments, the prodrug (e.g., a compound of Formula I, or Compound 1, Compound 2, Compound 3, Compound 4, Compound 5, or Compound 17), or a pharmaceutically acceptable salt thereof, is administered with food, twice daily. In some embodiments, the prodrug, or a pharmaceutically acceptable salt thereof, is administered with food, once daily. In some embodiments, the prodrug, or a pharmaceutically acceptable salt thereof, is administered with food, in a total daily dosage of about 75 mg to about 1,500 mg, about 75 mg to about 1,200 mg, about 75 mg to about 900 mg, about 75 mg to about 600 mg, about 75 mg to about 300 mg, about 150 mg to about 1,500 mg, about 150 mg to about 1,200 mg, about 150 mg to about 900 mg, about 150 mg to about 600 mg, about 150 mg to about 300 mg, about 300 mg to about 1,500 mg, about 300 mg to about 1,200 mg, about 300 mg to about 900 mg, about 300 mg to about 600 mg, about 600 mg to about 1,500 mg, about 600 mg to about 1,200 mg, or about 600 mg to about 900 mg, on a free base basis. In some embodiments, the prodrug, or a pharmaceutically acceptable salt thereof, is administered with food, in a total daily dosage of about 75 mg, about 150 mg, about 300 mg, about 600 mg, about 900 mg, or about 1,200 mg, on a free base basis.

Also provided herein is a method of treating IBD in a patient in need thereof, including administering to the patient a prodrug of Compound A or a pharmaceutically acceptable salt of the prodrug, wherein the prodrug, or a pharmaceutically acceptable salt thereof, is administered in a first dosage for a first period, and then in a second dosage for a second period. The first dosage is about 150 mg/dose to about 1,500 mg/dose on a free base basis, and the first period is up to about 24 weeks. In some embodiments, the method includes administering to the patient a prodrug of Compound A, in a first dosage of about 150 mg/dose to about 1,500 mg/dose, for a first period of up to about 24 weeks, and then in a second dosage for a second period. In some embodiments, the IBD is UC or CD.

In some embodiments, the prodrug administered in the first dosage and second dosage is a compound of Formula I, or a pharmaceutically acceptable salt thereof, wherein
R⁶ is —C(O)O—R¹⁶—OP(O)(OR¹²)₂, —C(O)—R¹⁶—OP(O)(OR¹²)₂, —R¹⁶—OP(O)(OR¹²)₂, —C(O)O—R¹⁶—OR¹⁷, —C(O)O—R¹⁶—OH, —C(O)O—R¹⁶—OC(O)R¹⁷, —C(O)—C(O)OR¹², or —C(O)O—R¹⁶—OC(O)R¹⁷NH₂;
each R¹² is independently hydrogen or C₁₋₉ alkyl;
R¹⁶ is —C₁₋₃ alkyl or cyclopropyl, each optionally substituted with one to four C₁₋₃ alkyl or cyclopropyl; and
R¹⁷ is C₁₋₉ alkyl, cycloalkyl, or heterocyclyl, each optionally substituted with one to three R¹⁶.

In certain such embodiments of Formula I, R⁶ is —C(O)O—R¹⁶—OP(O)(OR¹²)₂. In some embodiments, each R¹² is hydrogen. In some embodiments, R¹⁶ is C₁₋₃ alkyl. In some embodiments, R⁶ is

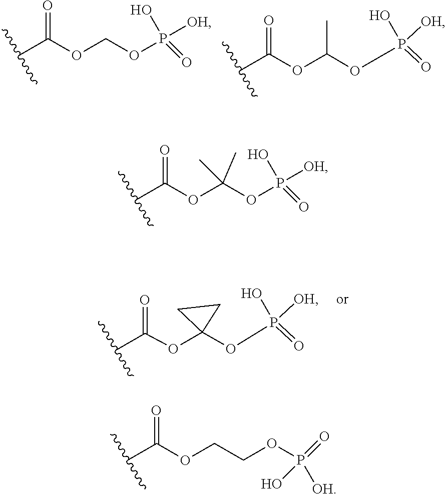 [SMILES: *C(=O)OCOP(=O)(O)O ; *C(=O)OC(C)OP(=O)(O)O ; *C(=O)OC(C)(C)OP(=O)(O)O ; *C(=O)OC1(OP(=O)(O)O)CC1 ; *C(=O)OCCOP(=O)(O)O]

In some embodiments, R⁶ is

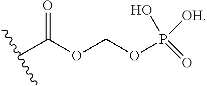 [SMILES: *C(=O)OCOP(=O)(O)O]

In some embodiments, R⁶ is

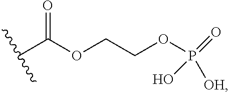 [SMILES: *C(=O)OCCOP(=O)(O)O]

In some embodiments, the prodrug administered in the first dosage and second dosage is Compound 1, Compound 2, Compound 3, Compound 4, Compound 5, or Compound 17, or a pharmaceutically acceptable salt thereof. In some embodiments, the prodrug administered in the first dosage and second dosage is Compound 1. For example, in certain such embodiments, the method comprises administering to the patient a prodrug which is Compound 1, in a first dosage of about 150 mg/dose to about 1,500 mg/dose, for a first period of up to about 24 weeks, and then in a second dosage for a second period.

In some embodiments, the first dosage is about 75 mg/dose to about 900 mg/dose, about 75 mg/dose to about 600 mg/dose, about 75 mg/dose to about 300 mg/dose, about 150 mg/dose to about 1,500 mg/dose, about 150 mg/dose to about 900 mg/dose, about 150 mg/dose to about 600 mg/dose, about 150 mg/dose to about 300 mg/dose, about 300 mg/dose to about 1,500 mg/dose, about 300 mg/dose to about 900 mg/dose, or about 300 mg/dose to about 600 mg/dose, on a free base basis. In some embodiments, the first dosage is about 75 mg/dose, 150 mg/dose, about 300 mg/dose, about 600 mg/dose, or about 900 mg/dose, on a free base basis. In some embodiments, the first dosage is administered twice daily. In some embodiments, the first dosage is administered once daily.

In some embodiments, the first period is up to about 22 weeks, up to about 20 weeks, up to about 18 weeks, up to about 16 weeks, up to about 14 weeks, up to about 12 weeks, up to about 10 weeks, up to about 8 weeks, up to about 6 weeks, up to about 4 weeks, or up to about 2 weeks. In some embodiments, the first period is about 2 weeks to about 24 weeks, about 2 weeks to about 20 weeks, about 2 weeks to about 16 weeks, about 2 weeks to about 12 weeks, about 4 weeks to about 24 weeks, about 4 weeks to about 20 weeks, about 4 weeks to about 16 weeks, about 4 weeks to about 12 weeks, about 8 weeks to about 24 weeks, about 8 weeks to about 20 weeks, about 8 weeks to about 16 weeks, about 8 weeks about 12 weeks, about 12 weeks to about 24 weeks, about 12 weeks to about 20 weeks, or about 12 weeks to about 16 weeks. In some embodiments, the first period is about 4 weeks, about 8 weeks, about 12 weeks, about 16 weeks, or about 20 weeks.

In some embodiments, the second dosage is less than or equal to the first dosage. In some embodiments, the second dosage is less than the first dosage. In some embodiments, the second dosage is greater than or equal to the first dosage. In some embodiments, the second dosage is equal to the first dosage.

In some embodiments, the second dosage is selected based on a response of the patient to the first dosage after the first period. For example, in some embodiments, the IBD is UC, and a response of the patient to the first dosage is determined based on stool frequency, rectal bleeding, endoscopic findings, or any combination thereof, after the first period. In some embodiments, the second dosage is selected for administration to a patient that had an adequate response to the first dosage, e.g., based on stool frequency, rectal bleeding, and endoscopic findings after the first period. In some embodiments, the second dosage is selected for administration to a patient had had an inadequate response to the first dosage, e.g., based on stool frequency, rectal bleeding, and endoscopic findings after the first period.

In some embodiments, the patient had an adequate response to the first dosage, and the second dosage is less than or equal to the first dosage. In certain such embodiments, the second dosage is about 75 mg/dose to about 600 mg/dose, about 75 mg/dose to about 300 mg/dose, about 150 mg/dose to about 600 mg/dose, about 150 mg/dose to about 300 mg/dose, or about 300 mg/dose to about 600 mg/dose, on a free base basis. In certain such embodiments, the second dosage is about 75 mg/dose, 150 mg/dose, or about 300 mg/dose, or about 600 mg/dose, on a free base basis. In certain such embodiments, the second dosage is administered twice daily. In certain such embodiments, the second dosage is administered once daily.

In some embodiments, the patient did not have an adequate response to the first dosage, and the second dosage is greater than or equal to the first dosage. In certain such embodiments, the second dosage is about 150 mg/dose to about 900 mg/dose, about 150 mg/dose to about 600 mg/dose, about 150 mg/dose to about 300 mg/dose, about 300 mg/dose to about 900 mg/dose, or about 300 mg/dose to about 600 mg/dose, on a free base basis. In certain such embodiments, the second dosage is about 150 mg/dose, about 300 mg/dose, about 600 mg/dose, or about 900 mg/dose, on a free base basis. In certain such embodiments, the second dosage is administered twice daily. In certain such embodiments, the second dosage is administered once daily.

In some embodiments, the second period is at least about 2 weeks, at least about 4 weeks, at least about 8 weeks, at least about 12 weeks, at least about 16 weeks, at least about 20 weeks, or at least about 24 weeks.

In some embodiments, the method includes administering a prodrug which is Compound 1 to the patient in a first dosage of about 600 mg/dose for a first period of up to about 24 weeks, determining that the patient achieved an adequate response to the first dosage after the first period, and administering the prodrug in a second dosage of about 300 mg/dose, for a second period. In certain such embodiments, the prodrug is administered once daily. In certain such embodiments, the first period is about 12 weeks. In certain such embodiments, the IBD is UC.

In some embodiments, the method includes administering a prodrug which is Compound 1 to the patient in a first dosage of about 600 mg/dose for a first period of up to about 24 weeks, determining that the patient did not achieve an adequate response to the first dosage after the first period, and administering the prodrug in a second dosage of about 600 mg/dose, for a second period. In certain such embodiments, the prodrug is administered once daily. In certain such embodiments, the first period is about 12 weeks. In certain such embodiments, the IBD is UC.

In some embodiments, the method includes administering a prodrug which is Compound 1 to the patient in a first dosage of about 600 mg/dose for a first period of up to about 24 weeks, determining that the patient did not achieve an adequate response to the first dosage after the first period, and administering the prodrug in a second dosage of about 300 mg/dose, for a second period. In certain such embodiments, the prodrug is administered once daily. In certain such embodiments, the first period is about 12 weeks. In certain such embodiments, the IBD is UC.

In some embodiments, the method includes administering a prodrug which is Compound 1 to the patient in a first dosage of about 300 mg/dose for a first period of up to about 24 weeks, determining that the patient achieved an adequate response to the first dosage after the first period, and administering the prodrug in a second dosage of about 300 mg/dose, for a second period. In certain such embodiments, the prodrug is administered once daily. In certain such embodiments, the first period is about 12 weeks. In certain such embodiments, the IBD is UC.

In some embodiments, the method includes administering a prodrug which is Compound 1 to the patient in a first dosage of about 300 mg/dose for a first period of up to about 24 weeks, determining that the patient achieved an adequate response to the first dosage after the first period, and administering the prodrug in a second dosage of about 150 mg/dose, for a second period. In certain such embodiments, the prodrug is administered once daily. In certain such embodiments, the first period is about 12 weeks. In certain such embodiments, the IBD is UC.

In some embodiments, the method includes administering a prodrug which is Compound 1 to the patient in a first dosage of about 300 mg/dose for a first period of up to about 24 weeks, determining that the patient did not achieve an adequate response to the first dosage after the first period, and administering the prodrug in a second dosage of about 300 mg/dose, for a second period. In certain such embodiments, the prodrug is administered once daily. In certain such embodiments, the first period is about 12 weeks. In certain such embodiments, the IBD is UC.

In some embodiments, the method includes administering a prodrug which is Compound 1 to the patient in a first dosage of about 150 mg/dose for a first period of up to about 24 weeks, determining that the patient achieved an adequate response to the first dosage after the first period, and administering the prodrug in a second dosage of about 150 mg/dose, for a second period. In certain such embodiments, the prodrug is administered once daily. In certain such embodiments, the first period is about 12 weeks. In certain such embodiments, the IBD is UC.

In some embodiments, the method includes administering a prodrug which is Compound 1 to the patient in a first dosage of about 150 mg/dose for a first period of up to about 24 weeks, determining that the patient did not achieve an adequate response to the first dosage after the first period, and administering the prodrug in a second dosage of about 300 mg/dose, for a second period. In certain such embodiments, the prodrug is administered once daily. In certain such embodiments, the first period is about 12 weeks. In certain such embodiments, the IBD is UC.

In some embodiments, the method includes administering Compound 1 or a pharmaceutically acceptable salt thereof to the patient in a first dosage of about 600 mg/dose, or about 300 mg/dose, or about 150 mg/dose for a first period of up to about 24 weeks, and then administering Compound 1 or a pharmaceutically acceptable salt thereof in a second dosage of about 600 mg/dose, about 300 mg/dose, or about 150 mg/dose for a second period. In certain such embodiments, the Compound 1 or pharmaceutically acceptable salt thereof is administered once daily. In certain such embodiments, the first period is about 12 weeks. In certain such embodiments, the IBD is UC. In certain such embodiments, the patient is a human. In certain embodiments, the first dosage is 600 mg/dose and the second dosage is 300 mg/dose. In certain embodiments, the first dosage is 300 mg/dose and the second dosage is 300 mg/dose. In certain embodiments, the first dosage is 600 mg/dose and the second dosage is 150 mg/dose. In certain embodiments, the first dosage is 300 mg/dose and the second dosage is 150 mg/dose. In certain embodiments, the first dosage is 150 mg/dose and the second dosage is 150 mg/dose.

Also provided herein is a method of treating IBD in a patient in need thereof, including administering to the patient a pharmaceutical composition or oral dosage form described herein. In certain such embodiments, the IBD is UC or CD.

### Pharmaceutical Compositions and Oral Dosage Forms

Also provided herein is a pharmaceutical composition including a prodrug of Compound A or a pharmaceutically acceptable salt of the prodrug, and further including a diluent, a disintegrant, or a lubricant, or any combination thereof. In some embodiments, the pharmaceutical composition includes a prodrug of Compound A, and further includes a diluent, a disintegrant, or a lubricant, or any combination thereof.

In some embodiments, the pharmaceutical composition includes a prodrug which is a compound of Formula I, or a pharmaceutically acceptable salt thereof, wherein
R⁶ is —C(O)O—R¹⁶—OP(O)(OR¹²)₂, —C(O)—R¹⁶—OP(O)(OR¹²)₂, —R¹⁶—OP(O)(OR¹²)₂, —C(O)O—R¹⁶—OR¹⁷, —C(O)O—R¹⁶—OH, —C(O)O—R¹⁶—OC(O)R¹⁷, —C(O)—C(O)OR¹², or —C(O)O—R¹⁶—OC(O)R¹⁷NH₂;
each R¹² is independently hydrogen or C₁₋₉ alkyl;
R¹⁶ is —C₁₋₃ alkyl or cyclopropyl, each optionally substituted with one to four C₁₋₃ alkyl or cyclopropyl; and
R¹⁷ is C₁₋₉ alkyl, cycloalkyl, or heterocyclyl, each optionally substituted with one to three R¹⁶.

In certain such embodiments of Formula I, R⁶ is —C(O)O—R¹⁶—OP(O)(OR¹²)₂. In some embodiments, each R¹² is hydrogen. In some embodiments, R¹⁶ is C₁₋₃ alkyl. In some embodiments, R⁶ is

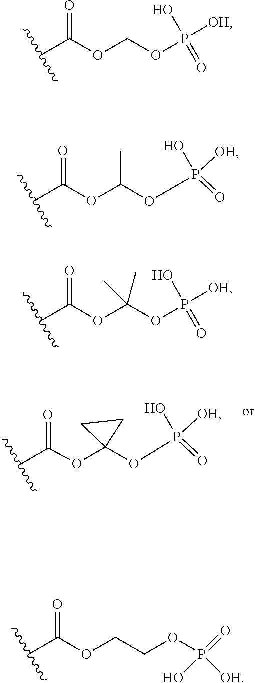 [SMILES: *C(=O)OCOP(=O)(O)O ; *C(=O)OC(C)OP(=O)(O)O ; *C(=O)OC(C)(C)OP(=O)(O)O ; *C(=O)OC1(OP(=O)(O)O)CC1 ; *C(=O)OCCOP(=O)(O)O]

In some embodiments, R⁶ is

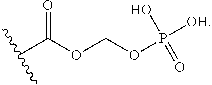 [SMILES: *C(=O)OCOP(=O)(O)O]

In some embodiments, R⁶ is

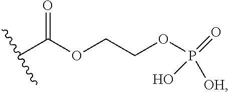 [SMILES: *C(=O)OCCOP(=O)(O)O]

In some embodiments, the pharmaceutical composition includes a prodrug which is Compound 1, Compound 2, Compound 3, Compound 4, Compound 5, or Compound 17, or a pharmaceutically acceptable salt thereof. In some embodiments, the pharmaceutical composition includes a prodrug which is Compound 1.

In some embodiments, the pharmaceutical composition includes a diluent, and the diluent includes lactose, lactose monohydrate, mannitol, isomalt, sucrose, dextrose, sorbitol, microcrystalline cellulose, silicified microcrystalline cellulose, acidified cellulose, colloidal silica, dicalcium phosphate dihydrate, calcium carbonate, or any combination thereof. In some embodiments, the pharmaceutical composition includes lactose monohydrate, microcrystalline cellulose, or both.

In some embodiments, the pharmaceutical composition includes a disintegrant, and the disintegrant includes croscarmellose sodium, crospovidone, starch (e.g., partially pregeletanized maize starch), cellulose, low substituted hydroxypropyl cellulose, alginic acid, sodium starch glycolate, or any combination thereof. In some embodiments, the pharmaceutical composition includes crospovidone.

In some embodiments, the pharmaceutical composition includes a lubricant, and the lubricant includes magnesium stearate, calcium stearate, stearic acid (stearin), talc, starch, fumed silica, hydrogenated oil, polyethylene glycol, sodium stearyl fumarate, glyceryl behenate, or any combination thereof. In some embodiments, the pharmaceutical composition includes magnesium stearate.

In some embodiments, the pharmaceutical composition is in oral dosage form. In some embodiments, the dosage form includes about 75 mg to about 600 mg, about 75 mg to about 300 mg, about 150 mg to about 600 mg, or about 15 mg to about 300 mg of the prodrug, or a pharmaceutically acceptable salt thereof, on a free base basis. In some embodiments, the dosage form includes about 150 mg or about 300 mg of the prodrug, or a pharmaceutically acceptable salt thereof, on a free base basis.

In some embodiments, the dosage form is a tablet. In certain such embodiments, the tablet includes a film coating. In some embodiments, the film coating includes a film-forming polymer, a pigment/opacifier, a plasticizer, or a filler, or any combination thereof.

In some embodiments, the film coating includes a film-forming polymer, and the film-forming polymer includes hydroxypropyl methylcellulose (hypromellose), sodium carboxymethyl cellulose, polyvinyl alcohol, methacrylic acid copolymer, or any combination thereof. In some embodiments, the film coating includes polyvinyl alcohol.

In some embodiments, the film coating includes a pigment/opacifier, and the pigment/opacifier includes aluminum flakes, iron oxides, titanium dioxide (titania), a natural color, or any combination thereof. In some embodiments, the film coating includes titanium dioxide.

In some embodiments, the film coating includes a plasticizer, and the plasticizer includes polyethylene glycol, glycerin, triethyl citrate, diethyl phthalate, or any combination thereof. In some embodiments, the film coating includes polyethylene glycol.

In some embodiments, the film coating includes a filler, and the filler includes talc, fumed silica, bentonite, an edible hydrogenated vegetable oil, or any combination thereof. In some embodiments, the film coating includes talc.

Also provided herein is an oral dosage form including a prodrug of Compound A or a pharmaceutically acceptable salt of the prodrug, and a pharmaceutically acceptable excipient, formulated to achieve one or more pharmacokinetic characteristics selected from:
an arithmetic mean steady-state AUCₜₐᵤ of Compound A of about 10 h*μg/mL to about 80 h*μg/mL;
an arithmetic mean steady-state Cₘₐₓ of Compound A of about 0.9 g/mL to about 9 μg/mL;
a median steady-state Tₘₐₓ of Compound A of about 1.5 hours to about 6 hours; and
a median steady-state t_{1/2} of Compound A of about 18 hours to about 30 hours,
   upon daily oral administration of the dosage form to a human patient in a fasted state. In some embodiments, the oral dosage form is formulated to achieve an arithmetic mean steady-state AUCₜₐᵤ of Compound A of about 10 h*μg/mL to about 80 h*μg/mL upon daily oral administration of the dosage form to a human patient in a fasted state. In some embodiments, the oral dosage form is formulated to achieve an arithmetic mean steady-state Cₘₐₓ of Compound A of about 0.9 g/mL to about 9 g/mL upon daily oral administration of the dosage form to a human patient in a fasted state. In some embodiments, the oral dosage form is formulated to achieve a median steady-state Tₘₐₓ of Compound A of about 1.5 hours to about 6 hours upon daily oral administration of the dosage form to a human patient in a fasted state. In some embodiments, the oral dosage form is formulated to achieve a median steady-state t_{1/2} of Compound A of about 18 hours to about 30 hours upon daily oral administration of the dosage form to a human patient in a fasted state.

In some embodiments, the oral dosage form includes a prodrug of Compound A and a pharmaceutically acceptable excipient, and is formulated to achieve the one or more pharmacokinetic characteristics upon daily oral administration of the dosage form to a human patient in a fasted state.

In some embodiments, the oral dosage form includes a prodrug which is a compound of Formula I, or a pharmaceutically acceptable salt thereof, wherein
R⁶ is —C(O)O—R¹⁶—OP(O)(OR¹²)₂, —C(O)—R¹⁶—OP(O)(OR¹²)₂, —R¹⁶—OP(O)(OR¹²)₂, —C(O)O—R¹⁶—OR¹⁷, —C(O)O—R¹⁶—OH, —C(O)O—R¹⁶—OC(O)R¹⁷, —C(O)—C(O)OR¹², or —C(O)O—R¹⁶—OC(O)R¹⁷NH₂;
each R¹² is independently hydrogen or C₁₋₉ alkyl;
R¹⁶ is —C₁₋₃ alkyl or cyclopropyl, each optionally substituted with one to four C₁₋₃ alkyl or cyclopropyl; and
R¹⁷ is C₁₋₉ alkyl, cycloalkyl, or heterocyclyl, each optionally substituted with one to three R¹⁶.

In certain such embodiments of Formula I, R⁶ is —C(O)O—R¹⁶—OP(O)(OR¹²)₂. In some embodiments, each R¹² is hydrogen. In some embodiments, R¹⁶ is C₁₋₃ alkyl. In some embodiments, R⁶ is

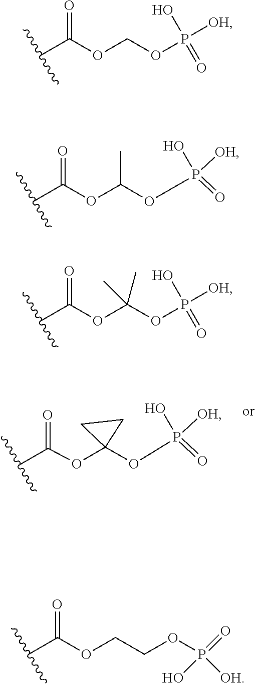 [SMILES: *C(=O)OCOP(=O)(O)O ; *C(=O)OC(C)OP(=O)(O)O ; *C(=O)OC(C)(C)OP(=O)(O)O ; *C(=O)OC1(OP(=O)(O)O)CC1 ; *C(=O)OCCOP(=O)(O)O]

In some embodiments, R⁶ is

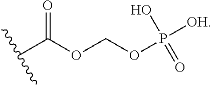 [SMILES: *C(=O)OCOP(=O)(O)O]

In some embodiments, R⁶ is

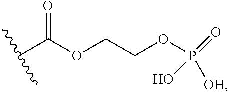 [SMILES: *C(=O)OCCOP(=O)(O)O]

In some embodiments, the oral dosage form includes a prodrug which is Compound 1, Compound 2, Compound 3, Compound 4, Compound 5, or Compound 17, or a pharmaceutically acceptable salt thereof. In some embodiments, the oral dosage form includes a prodrug which is Compound 1.

In some embodiments, the oral dosage form is formulated to achieve an arithmetic mean steady-state AUCₜₐᵤ of Compound A of about 10 h*μg/mL to about 70 h*μg/mL, about 10 h*μg/mL to about 65 h*μg/mL, about 10 h*μg/mL to about 60 h*μg/mL, about 20 h*μg/mL to about 80 h*μg/mL, about 20 h*μg/mL to about 70 h*μg/mL, about 20 h*μg/mL to about 65 h*μg/mL, about 20 h*μg/mL to about 60 h*μg/mL, about 35 h*μg/mL to about 80 h*μg/mL, about 35 h*μg/mL to about 70 h*μg/mL, about 35 h*μg/mL to about 65 h*μg/mL, about 35 h*μg/mL to about 60 h*μg/mL, about 40 h*μg/mL to about 80 h*μg/mL, about 40 h*μg/mL to about 70 h*μg/mL, about 40 h*μg/mL to about 65 h*μg/mL, or about 40 h*μg/mL to about 60 h*μg/mL, upon daily oral administration of the dosage form to a human patient in a fasted state. In some embodiments, the oral dosage form is formulated to achieve an arithmetic mean steady-state AUCₜₐᵤ of Compound A of about 10.5 h*μg/mL, about 37.4 h*μg/mL, about 55.1 h*μg/mL, about 62.2 h*μg/mL, or about 77.5 h*μg/mL, upon daily oral administration of the dosage form to a human patient in a fasted state.

In some embodiments, the oral dosage form is formulated to achieve an arithmetic mean steady-state Cₘₐₓ of Compound A of about 0.9 g/mL to about 7 g/mL, about 0.9 g/mL to about 6 g/mL, about 0.9 g/mL to about 5 g/mL, about 1.5 g/mL to about 9 g/mL, about 1.5 g/mL to about 7 g/mL, about 1.5 g/mL to about 6 g/mL, about 1.5 g/mL to about 5 μg/mL, about 2.5 g/mL to about 9 g/mL, about 2.5 g/mL to about 7 g/mL, about 2.5 μg/mL to about 6 g/mL, about 2.5 g/mL to about 5 g/mL, about 3 g/mL to about 9 g/mL, about 3 g/mL to about 7 g/mL, about 3 g/mL to about 6 g/mL, or about 3 g/mL to about 5 g/mL, upon daily oral administration of the dosage form to a human patient in a fasted state. In some embodiments, the oral dosage form is formulated to achieve an arithmetic mean steady-state Cₘₐₓ of Compound A of about 0.1 g/mL, about 3.2 g/mL, about 3.8 g/mL, about 5 μg/mL, or about 8.6 g/mL, upon daily oral administration of the dosage form to a human patient in a fasted state.

In some embodiments, the oral dosage form is formulated to achieve a median steady-state Tₘₐₓ of Compound A of about 1.5 hours to about 5 hours, about 1.5 hours to about 4 hours, about 2 hours to about 6 hours, about 2 hours to about 5 hours, about 2 hours to about 4 hours, about 2 hours to about 6 hours, about 2 hours to about 5 hours, or about 2 hours to about 4 hours, upon daily oral administration of the dosage form to a human patient in a fasted state. In some embodiments, the oral dosage form is formulated to achieve a median steady-state Tₘₐₓ of Compound A of about 2 hours, about 3 hours, or about 4 hours, upon daily oral administration of the dosage form to a human patient in a fasted state.

In some embodiments, the oral dosage form is formulated to achieve a median steady-state t_{1/2} of Compound A of about 18 hours to about 29 hours, about 18 hours to about 28 hours, about 19 hours to about 30 hours, about 19 hours to about 29 hours, about 19 hours to about 28 hours, about 21 hours to about 30 hours, about 21 hours to about 29 hours, or about 21 hours to about 28 hours, upon daily oral administration of the dosage form to a human patient in a fasted state. In some embodiments, the oral dosage form is formulated to achieve a median steady-state t_{1/2} of Compound A of about 19 hours, about 22 hours, about 28 hours, or about 29 hours, upon daily oral administration of the dosage form to a human patient in a fasted state.

In some embodiments, the pharmaceutically acceptable excipient includes a diluent, a disintegrant, a binder, a lubricant, or any combination thereof. In some embodiments, the pharmaceutically acceptable excipient includes a diluent described herein, a disintegrant described herein, a lubricant described herein, or any combination thereof. In certain such embodiments, the pharmaceutically acceptable excipient includes lactose monohydrate, crospovidone, or magnesium stearate, or any combination thereof.

In some embodiments, the oral dosage form is a tablet. In certain such embodiments, the tablet includes a film coating. In some embodiments, the film coating includes a film-forming polymer described herein, a pigment/opacifier described herein, a plasticizer described herein, a filler described herein, or any combination thereof. In certain such embodiments, the film coating includes polyvinyl alcohol, titanium dioxide, polyethylene glycol, talc, or any combination thereof. In certain such embodiments, the film coating includes polyvinyl alcohol, polyethylene glycol, talc, or any combination thereof.

In some embodiments, the oral dosage form includes about 75 mg to about 600 mg, about 75 mg to about 300 mg, about 150 mg to about 600 mg, or about 15 mg to about 300 mg of the prodrug, or a pharmaceutically acceptable salt thereof, on a free base basis. In some embodiments, the dosage form includes about 150 mg or about 300 mg of the prodrug, or a pharmaceutically acceptable salt thereof, on a free base basis.

### EXAMPLES

### Example 1. Dose-Escalation Study in Healthy Participants

A 2-part, single- and multiple-ascending dose sponsor-unblinded Phase 1 study was conducted for Cohorts 1-9 to evaluate the safety, tolerability, PK, and PD of Compound 1 in healthy participants. Up to 72 participants ages 18 to 55 years (inclusive) and having body mass index (BMI) of ≥19.0 and ≤30.0 kg/m² were enrolled. For Cohort 9, participants were homozygous for the UGT1A1*28 allele (UGT1A1 *28/*28: TA7/TA7).

### Part A: Cohorts 1-4; Single Ascending Dose

Part A proceeded in 4 staggered, dose-escalation cohorts. Within each of Cohorts 1-4, 8 unique participants were randomized 3:1 to receive blinded Compound 1 (N=6) or placebo-to-match (PTM) (N=2). The dose of Compound 1 for all adaptive cohorts (Cohorts 2, 3, and 4) was determined based on cumulative safety and available PK data through a minimum of Day 3 from previous cohorts, not exceeding the specified upper dose limit (Table 2). Dosing in Part A was completed prior to proceeding with Part B.

TABLE 2

| | Single Ascending Dose |
|---|---|
| Cohort | Day 1 |
| 1 | 75 mg Compound 1 or PTM (3 × 25 mg), |
| | single dose, fasted |
| 2 | 300 mg Compound 1 or PTM (2 × 150 mg), |
| | single dose, fasted |
| 3 | 900 mg Compound 1 or PTM (6 × 150 mg), |
| | single dose, fasted |
| 4 | 1500 mg Compound 1 or PTM (10 × 150 mg), |
| | single dose, fasted |

### Part B: Cohorts 5-9; Multiple Ascending Dose

Part B proceeded in up to 5 staggered, dose-escalation cohorts. Within each of Cohorts 5-9, 8 unique participants were randomized 3:1 to receive blinded Compound 1 (N=6) or PTM (N=2). Start of multiple dosing within Adaptive Cohort 5 was determined by review of the cumulative safety and PK data through at least Day 3 of the final cohort in Part A. Initiation of subsequent multiple-dose cohorts at a higher dose (Cohorts 6, 7, 8, and 9) was determined based on cumulative safety through at least Day 10 from all participants enrolled in the previous multiple-dose cohort (Table 3).

TABLE 3

| | Multiple Ascending Dose |
|---|---|
| Cohort | (Day 1-10) |
| 5 | 75 mg Compound 1 or PTM (3 × 25 mg), once daily, fasted |
| 6 | 300 mg Compound 1 or PTM (2 × 150 mg), once daily, fasted |
| 7 | 900 mg Compound 1 or PTM (6 × 150 mg), once daily, fasted |
| 8 | 600 mg Compound 1 or PTM (4 × 150 mg), twice daily, fasted |
| 9^{a} | 600 mg Compound 1 or PTM (4 × 150 mg), once daily, fasted |

^{a}Cohort 9 included only the participants that were homozygous for UGT1A1*28 allele (UGT1A1 *28/*28: TA7/TA7)

Compound 1 was administered as tablets in strengths of 25 mg or 150 mg. In addition to the active ingredient, Compound 1 tablets also contained lactose monohydrate, crospovidone, magnesium stearate, polyvinyl alcohol, titanium dioxide, polyethylene glycol, and talc.

PTM was administered as tablets identical in size, shape, color, and appearance to corresponding active Compound 1 tablets. PTM tablets contained lactose monohydrate, microcrystalline cellulose, croscarmellose sodium, magnesium stearate, polyvinyl alcohol, titanium dioxide, polyethylene glycol, and talc.

For Cohorts 1-4, plasma PK sampling occurred relative to the dose of Compound 1 at the following time points:
Day 1: 0 (pre-dose, ≤5 min prior to dosing), 0.25, 0.5, 1, 1.5, 2, 3, 4, 6, 8, 12, 18, 24, 36, 48, 72, 96, and 120 hours post-dose
   A single plasma PK sample was collected at early termination (ET), if applicable.

For Cohorts 1-4, whole blood PD sampling occurred relative to the dose of Compound 1 at the following time points:
Day 1: 0 pre-dose (≤5 min prior to dosing), 1, 2, 3, 4, 8, 12, 24 and 36 hours post-dose

For Cohorts 5-9, plasma PK sampling occurred relative to the dose of Compound 1 at the following time points:
Day 1 (Cohorts 8 and 9): pre-dose (≤5 min before dose), 0.5, 1, 2, 3, 4, 6, 8, 12, 13, 14, 15, 16, 18, and 20 hours post-dose (relative to AM dose)
Day 10 (Cohorts 5-9): pre-dose (≤5 min before dose), 0.25, 0.5, 1, 1.5, 2, 3, 4, 6, 8, 12, 18, 24, 36, 48, 72, 96, and 120 hours post-dose
A pre-dose sample was collected on Days 2, 4, 7, and 8.
   A single plasma PK sample was collected at early termination (ET), if applicable.

For Cohorts 5-9, whole blood PD sampling occurred relative to the dose of Compound 1 at the following time points:
Day 1: 0 pre-dose (≤5 min prior to dosing), 1, 2, 3, 4, 8, 12, and 24 hours post-dose
Day 10: 0 pre-dose (≤5 min prior to dosing), 1, 2, 3, 4, 8, 12, 24, 36, 48, 72, 96, and 120 hours post-dose

Concentrations of Compound 1, and its respective metabolites Compound A and Compound B in plasma PK samples were determined by validated high-performance liquid chromatography-tandem mass spectrometry (LC-MS/MS) bioanalytical methods.

Whole blood PD Samples were stimulated ex vivo with lipopolysaccharide (LPS) or phosphate buffered saline (PBS), and the phosphorylation of extracellular signal-related kinase (pERK) was detected in monocytes by flow cytometry.

Safety assessments included monitoring of adverse events (AEs) and concomitant medications, clinical laboratory analyses, vital sign measurements, electrocardiograms (ECGs), and physical examinations.

### Pharmacokinetic Results

Pharmacokinetic parameters for Compound 1 and its metabolites were determined from the plasma concentration-time profiles for all evaluable participants. The parameters are set forth in Table 4, below.

TABLE 4

| Parameter | Definition |
|---|---|
| Cₘₐₓ | maximum observed concentration of drug |
| Cₘₐₓ/D | dose-normalized Cₘₐₓ |
| Cₜₐᵤ | observed drug concentration at the end of the dosing interval |
| AUC_{inf} | area under the concentration versus time curve extrapolated |
| | to infinite time, calculated as AUCₗₐₛₜ + (Cₗₐₛₜ/λ_{z}) |
| AUC_{inf}/D | dose-normalized AUC_{inf} |
| AUCₗₐₛₜ | area under the concentration versus time curve from time |
| | zero to the last quantifiable concentration |
| AUCₜₐᵤ | area under the concentration versus time curve over the |
| | dosing interval tau |
| AUCₜₐᵤ/D | dose-normalized AUCₜₐᵤ |
| Tₘₐₓ | time (observed time point) of Cₘₐₓ |
| t_{1/2} | terminal elimination half-life |

Compound 1 was administered under fasting conditions following an overnight fast (no food or drinks except water) for at least 10 hours; food could be consumed 4 hours after administration of Compound 1.

Following single oral doses of Compound 1 (300 to 1500 mg) under such fasted conditions, Compound A plasma exposure (Cₘₐₓ, AUC_{inf}, and AUCₗₐₛₜ) increased in a dose-dependent manner in the Compound 1 300 to 900 mg dose range, with no further increase in exposure with dose escalation to 1500 mg. The median times at which Compound A reached maximal plasma concentrations (Tₘₐₓ) ranged from 3 to 4 hours post-dose. The median terminal elimination t_{1/2} ranged from 22 to 29 hours across the dose levels evaluated. Interindividual variability of single-dose Compound A Cₘₐₓ, AUC_{inf}, and AUCₗₐₛₜ was generally low to moderate, ranging from 23% to 63% CV.

Following multiple once-daily doses of Compound 1 (75 to 900 mg) or 600 mg Compound 1 twice daily under fasted conditions, Compound A steady-state plasma exposure (Cₘₐₓ and AUC) increased in a dose-dependent manner from 75 to 900 mg. PK parameters after administration of Compound 1 (75, 300, 600, or 900 mg) once daily or Compound 1 (600 mg) twice daily under fasted conditions on Day 1 (600 mg) and after 10 days of dosing (Day 10) are shown in Tables 5 and 6, below.

TABLE 5

| | | | |
|---|---|---|---|
| | Cohort 5 | Cohort 6 | Cohort 7 |
| Cpd A PK Parameter | 75 mg Cpd 1 QD | 300 mg Cpd 1 QD | 900 mg Cpd 1 QD |
| (Mean^{a} [% CV]) | (N = 5) | (N = 6) | (N = 6) |

Day 10

| | | | | | | |
|---|---|---|---|---|---|---|
| AUCₜₐᵤ | 10.5 | (27.2) | 37.4 | (30.7) | 62.2 | (29.6) |
| (h · μg/mL) ^{b} | | | | | | |
| Cₘₐₓ | 0.980 | (27.6) | 3.17 | (22.6) | 4.96 | (32.0) |
| (μg/mL) ^{b} | | | | | | |
| Cₜₐᵤ | 0.273 | (41.3) | 1.07 | (45.7) | 2.33 | (36.8) |
| (μg/mL) ^{b} | | | | | | |
| Tₘₐₓ | 3.00 | (2.00, 3.10) | 4.00 | (1.50, 4.00) | 3.01 | (2.00, 4.00) |
| (h)^{c} | | | | | | |
| t_{1/2} | 21.8 | (20.3, 31.8) | 19.0 | (13.9, 40.0) | 28.5 | (20.1, 39.1) |
| (h)^{c} | | | | | | |
| AUCₜₐᵤ/D | 0.141 | (27.2) | 0.125 | (30.7) | 0.0691 | (29.6) |
| (h · μg/mL) ^{b} | | | | | | |
| Cₘₐₓ/D | 0.0131 | (27.6) | 0.0106 | (22.6) | 0.0055 | (32.0) |
| (μg/mL) ^{b} | | | | | | |

BID = twice daily;
% CV = percentage coefficient of variation;
MAD = multiple ascending dose;
PK = pharmacokinetic(s);
QD = once daily;
UGTIA1 = uridine diphosphate glucuronosyltransferase 1A1
^{a}Means presented are unadjusted arithmetic means.
^{b} Values are for the morning dose (AM) in Cohort 8.
^{c}Values are presented as median (minimum, maximum).

TABLE 6

| | | |
|---|---|---|
| | | Cohort 9 |
| Cpd A PK | Cohort 8 | (UGT1A1 *28/*28) |
| Parameter | 600 mg Cpd 1 BID | 600 mg Cpd 1 QD |
| (Mean^{a} [% CV]) | (N = 6) | (N = 6) |

Day 1

| | | | | |
|---|---|---|---|---|
| AUCₜₐᵤ (h · μg/mL)^{b} | 15.9 | (18.9) | 25.6 | (15.2) |
| Cₘₐₓ (μg/mL)^{b} | 2.48 | (20.4) | 2.76 | (18.2) |
| Cₜₐᵤ (μg/mL)^{b} | 0.972 | (33.3) | 0.507 | (34.9) |
| Tₘₐₓ (h)^{c} | 2.50 | (1.12, 4.00) | 3.00 | (2.00, 4.00) |
| AUCₜₐᵤ/D (h · μg/mL)^{b} | 0.0264 | (18.9) | 0.0427 | (15.2) |
| Cₘₐₓ/D (μg/mL)^{b} | 0.0041 | (20.4) | 0.0046 | (18.2) |

Day 10

| | | | | |
|---|---|---|---|---|
| AUCₜₐᵤ (h · μg/mL)^{b} | 77.5 | (29.1) | 55.1 | (39.4) |
| Cₘₐₓ (μg/mL)^{b} | 8.64 | (33.0) | 3.82 | (33.0) |
| Cₜₐᵤ (μg/mL)^{b} | 5.65 | (24.1) | 1.70 | (46.0) |
| Tₘₐₓ (h)^{c} | 2.00 | (2.00, 6.00) | 3.00 | (2.00, 6.00) |
| t_{1/2} (h)^{c} | 21.8 | (13.0, 73.4) | 28.4 | (20.3, 43.7) |
| AUCₜₐᵤ/D (h · μg/mL)^{b} | 0.129 | (29.1) | 0.0918 | (39.4) |
| Cₘₐₓ/D (μg/mL)^{b} | 0.0144 | (33.0) | 0.0064 | (33.0) |

BID = twice daily;
% CV = percentage coefficient of variation;
MAD = multiple ascending dose;
PK = pharmacokinetic(s);
QD = once daily;
UGTIA1 = uridine diphosphate glucuronosyltransferase 1A1
^{a}Means presented are unadjusted arithmetic means.
^{b}Values are for the morning dose (AM) in Cohort 8.
^{c}Values are presented as median (minimum, maximum).

Compound A median Tₘₐₓ values ranged from 2 to 4 hours after Compound 1 dosing and Compound A median t_{1/2} values ranged from 19 to 28 hours. The variability of Compound A AUC and Cₘₐₓ parameters ranged from 14% to 39% CV, consistent with the single-dose cohorts. Compound A maximum plasma concentrations were achieved between 2.0 and 4.0 hours (median Tₘₐₓ) following Compound 1 administration.

Across the Compound 1 dose levels evaluated, Compound A exhibited a median t_{1/2} of 20 to 30 hours following single or multiple doses. Interindividual variability (% CV) was generally low to moderate across cohorts, ranging from 13% to 46% CV. Consistent with Compound A elimination t_{1/2}, Compound A exposure (AUC) accumulated approximately 2-fold following multiple dosing of 600 mg once daily. Steady-state exposures of Compound A were achieved by Day 7 of Compound 1 once-daily dosing.

### Pharmacodynamic Results

Pharmacodynamic changes after single and multiple doses of Compound 1 were also investigated by monitoring, in whole blood samples, stimulated with either lipopolysaccharide (LPS) or PBS for 15 minutes at 37° C. Changes to the phosphorylation of extracellular signal-regulated kinases (pERK) was measured in monocytes by flow cytometry, defined as CD15−CD14+.

Pharmacodynamic parameters for Compound 1 were calculated as percent change in pERK signal relative to Day 1 pre-dose (baseline) for all evaluable participants.

Single Ascending Doses: Following single oral doses of Compound 1 (300 to 1500 mg) under fasted conditions, a dose-dependent and reversible inhibition of LPS-stimulated pERK was observed, with peak inhibition at 3 hours post-dose for all Compound 1 dosages administered. At 3 hours post-last dose, the median change in pERK relative to baseline was 0.2%, −86.8%, −95.6%, −99.9%, and −99% for placebo, 75 mg Compound 1, 300 mg Compound 1, 900 mg Compound 1, and 1500 mg Compound 1, respectively. At 12 hours post-last dose, the median change in pERK relative to baseline was 9%, −51.3%, −76.6%, −92.1%, and −96.3% for placebo, 75 mg Compound 1, 300 mg Compound 1, 900 mg Compound 1, and 1500 mg Compound 1, respectively. The percent inhibition of pERK signal, by dose cohort and normalized to baseline, after a single dose of Compound 1 (75, 300, 900, or 1500 mg) is shown in Table 7, below.

TABLE 7
Summary statistics for pERK Signal, % Inhibition by
Dose Cohort, Normalized to Baseline (SAD Cohorts)

| | Compound 1 | Compound 1 | Compound 1 | Compound 1 | |
|---|---|---|---|---|---|
| | 75 mg | 300 mg | 900 mg | 1500 mg | |
| | Single | Single | Single | Single | |
| | Dose | Dose | Dose | Dose | Placebo |
| | (N = 4) | (N = 6) | (N = 6) | (N = 6) | (N = 8) |
| pERK Signal, % | | | | | |
| Inhibition | | | | | |
| at Day 1 | | | | | |
| Predose | | | | | |
| N | 4 | 6 | 6 | 6 | 8 |
| Mean (SD) | 0.0 (0.00) | 0.0 (0.00) | 0.0 (0.00) | 0.0 (0.00) | 0.0 (0.00) |
| Median | 0.0 | 0.0 | 0.0 | 0.0 | 0.0 |
| Q1, Q3 | 0.0, 0.0 | 0.0, 0.0 | 0.0, 0.0 | 0.0, 0.0 | 0.0, 0.0 |
| Min, Max | 0.0, 0.0 | 0.0, 0.0 | 0.0, 0.0 | 0.0, 0.0 | 0.0, 0.0 |
| P-value | 1.0000 | 1.0000 | 1.0000 | 1.0000 | NA |
| (Wilcoxon) | | | | | |
| P-value (JT) | NA | NA | NA | NA | NA |
| pERK Signal, % | | | | | |
| Inhibition | | | | | |
| at Day 1 - | | | | | |
| 1 hour | | | | | |
| N | 3 | 6 | 6 | 5 | 8 |
| Mean (SD) | 56.4 (13.04) | 32.2 (35.11) | 74.5 (38.94) | 74.8 (45.62) | 0.4 (7.15) |
| Median | 62.9 | 29.1 | 97.9 | 97.3 | −1.3 |
| Q1, Q3 | 41.4, 64.9 | 2.9, 34.9 | 25.4, 98.8 | 82.0, 100.0 | −3.6, 2.2 |
| Min, Max | 41.4, 64.9 | 0.3, 97.2 | 23.2, 103.7 | −5.7, 100.4 | −7.0, 15.8 |
| P-value | 0.0189 | 0.0118 | 0.0024 | 0.0673 | NA |
| (Wilcoxon) | | | | | |
| P-value (JT) | NA | NA | NA | NA | 0.00032 |
| pERK Signal, % | | | | | |
| Inhibition | | | | | |
| at Day 1 - | | | | | |
| 3 hours | | | | | |
| N | 4 | 6 | 6 | 6 | 8 |
| Mean (SD) | 82.7 (10.42) | 93.4 (5.08) | 99.8 (2.98) | 92.8 (16.61) | −0.3 (11.61) |
| Median | 86.8 | 95.6 | 99.9 | 99.0 | −0.2 |
| Q1, Q3 | 76.9, 88.4 | 90.0, 96.4 | 97.3, 101.4 | 97.7, 101.0 | −9.9, 8.7 |
| Min, Max | 67.2, 89.8 | 84.6, 98.1 | 96.0, 104.4 | 59.0, 101.1 | −16.8, 17.4 |
| P-value | 0.0085 | 0.0024 | 0.0024 | 0.0024 | NA |
| (Wilcoxon) | | | | | |
| P-value (JT) | NA | NA | NA | NA | 2.94E−7 |
| pERK Signal, % | | | | | |
| Inhibition | | | | | |
| at Day 1 - | | | | | |
| 12 hours | | | | | |
| N | 4 | 6 | 6 | 6 | 8 |
| Mean (SD) | 52.2 (6.53) | 76.0 (7.04) | 92.7 (3.10) | 93.0 (6.35) | −7.4 (15.91) |
| Median | 51.3 | 76.7 | 92.1 | 96.3 | −9.0 |
| Q1, Q3 | 47.0, 57.4 | 68.7, 81.6 | 90.8, 95.9 | 86.2, 97.8 | −20.1, 0.4 |
| Min, Max | 45.9, 60.4 | 68.0, 84.0 | 88.6, 96.6 | 83.7, 97.8 | −24.4, 22.5 |
| P-value | 0.0085 | 0.0024 | 0.0024 | 0.0024 | NA |
| (Wilcoxon) | | | | | |
| P-value (JT) | NA | NA | NA | NA | 2.38E−9 |
| pERK Signal, % | | | | | |
| Inhibition | | | | | |
| at Day 1 - | | | | | |
| 24 hours | | | | | |
| N | 3 | 6 | 6 | 6 | 8 |
| Mean (SD) | 33.6 (4.73) | 66.4 (2.19) | 83.6 (7.00) | 94.4 (2.50) | −2.2 (14.15) |
| Median | 33.6 | 65.3 | 85.8 | 94.2 | −2.5 |
| Q1, Q3 | 28.9, 38.3 | 65.0, 68.6 | 81.0, 87.3 | 92.3, 96.2 | −13.0, 8.9 |
| Min, Max | 28.9, 38.3 | 64.5, 69.7 | 70.9, 91.0 | 91.7, 98.2 | −22.6, 17.9 |
| P-value | 0.0189 | 0.0024 | 0.0024 | 0.0024 | NA |
| (Wilcoxon) | | | | | |
| P-value (JT) | NA | NA | NA | NA | 1.95−10 |

P-value (Wilcoxon): Wilcoxon Rank Sum test comparing treatment arms to pooled placebo.
P-value (JT): Jonckheere's Trend Test indicating probability of dose-dependent response.

Multiple Ascending Doses: Pharmacodynamic parameters after multiple doses of Compound 1 were also calculated as percent change in pERK signal relative to Day 1 pre-dose (baseline) for all evaluable participants.

On Day 10 of multiple once-daily doses of Compound 1 (75 to 900 mg) or 600 mg Compound 1 twice daily under fasted conditions, a trend in dose-dependent and reversible inhibition of LPS-stimulated pERK was observed. On Day 10, a maximum PD effect was observed between 2-4 hours post-last dose.

On Day 10 at 12 hours post-last dose, median percent change in pERK relative to baseline was 14.2%, −48.3%, −95.7%, −96.7%, and −100.2% for placebo, 75 mg Compound 1 QD, 300 mg Compound 1 QD, 900 mg Compound 1 QD, and 600 mg Compound 1 BID, respectively. On Day 10 at 24 hours post last dose, median percent change in pERK relative to baseline was −3.9%, −39.8%, −91.6%, −98.5%, and −101.5% for placebo, 75 mg Compound 1 QD, 300 mg Compound 1 QD, 900 mg Compound 1 QD, and 600 mg Compound 1 BID, respectively. The percent inhibition of pERK signal, by dose cohort and normalized to baseline, after multiple doses of Compound 1 (75, 300, and 900 mg QD and 600 mg BID) is shown in Table 8, below.

TABLE 8
Summary statistics for pERK Signal, % Inhibition by
Dose Cohort, Normalized to Baseline (MAD Cohorts)

| | Compound 1 | Compound 1 | Compound 1 | Compound 1 | |
|---|---|---|---|---|---|
| | 75 mg QD | 300 mg QD | 900 mg QD | 600 mg BID | |
| | Multiple | Multiple | Multiple | Multiple | |
| | Dose | Dose | Dose | Dose | Placebo |
| | (N = 5) | (N = 5) | (N = 6) | (N = 6) | (N = 8) |
| pERK Signal, % | | | | | |
| Inhibition | | | | | |
| at Day 1 | | | | | |
| Predose | | | | | |
| N | 5 | 5 | 6 | 6 | 8 |
| Mean (SD) | 0.0 (0.00) | 0.0 (0.00) | 0.0 (0.00) | 0.0 (0.00) | 0.0 (0.00) |
| Median | 0.0 | 0.0 | 0.0 | 0.0 | 0.0 |
| Q1, Q3 | 0.0, 0.0 | 0.0, 0.0 | 0.0, 0.0 | 0.0, 0.0 | 0.0, 0.0 |
| Min, Max | 0.0, 0.0 | 0.0, 0.0 | 0.0, 0.0 | 0.0, 0.0 | 0.0, 0.0 |
| P-value | 1.0000 | 1.0000 | 1.0000 | 1.0000 | NA |
| (Wilcoxon) | | | | | |
| P-value (JT) | NA | NA | NA | NA | NA |
| pERK Signal, % | | | | | |
| Inhibition | | | | | |
| at Day 1 | | | | | |
| 1 hour | | | | | |
| N | 5 | 5 | 6 | 6 | 7 |
| Mean (SD) | 47.9 (28.59) | 89.1 (11.30) | 88.9 (15.31) | 79.9 (15.78) | 23.4 (30.56) |
| Median | 44.9 | 93.8 | 95.0 | 74.3 | 3.6 |
| Q1, Q3 | 39.4, 67.2 | 88.7, 94.8 | 88.7, 97.9 | 68.6, 98.7 | −1.7, 52.9 |
| Min, Max | 6.6, 81.3 | 69.9, 98.4 | 58.5, 98.1 | 63.0, 100.3 | −7.7, 62.5 |
| P-value | 0.1939 | 0.0058 | 0.0053 | 0.0034 | NA |
| (Wilcoxon) | | | | | |
| P-value (JT) | NA | NA | NA | NA | 0.00017 |
| pERK Signal, % | | | | | |
| Inhibition | | | | | |
| at Day 1 | | | | | |
| 3 hours | | | | | |
| N | 5 | 5 | 6 | 6 | 7 |
| Mean (SD) | 67.5 (28.75) | 95.7 (2.92) | 99.8 (1.66) | 99.9 (5.48) | 25.1 (24.51) |
| Median | 79.6 | 97.1 | 99.6 | 99.9 | 16.1 |
| Q1, Q3 | 73.6, 82.2 | 95.4, 97.5 | 98.6, 100.1 | 94.1, 105.3 | 4.4, 52.2 |
| Min, Max | 16.6, 85.4 | 90.7, 97.7 | 98.2, 102.8 | 93.5, 106.6 | −1.5, 55.7 |
| P-value | 0.0230 | 0.0058 | 0.0034 | 0.0034 | NA |
| (Wilcoxon) | | | | | |
| P-value (JT) | NA | NA | NA | NA | 6.65E−8 |
| pERK Signal, % | | | | | |
| Inhibition | | | | | |
| at Day 1 | | | | | |
| 12 hours | | | | | |
| N | 5 | 5 | 6 | 6 | 8 |
| Mean (SD) | 26.1 (22.77) | 75.3 (5.34) | 95.2 (2.46) | 84.2 (16.33) | 5.3 (21.33) |
| Median | 32.4 | 74.3 | 95.3 | 91.0 | 8.8 |
| Q1, Q3 | 24.2, 39.6 | 73.0, 75.0 | 93.1, 97.7 | 76.4, 95.5 | −6.4, 24.0 |
| Min, Max | −11.9, 46.2 | 69.9, 84.2 | 91.8, 98.0 | 54.3, 96.8 | −35.1, 24.8 |
| P-value | 0.0923 | 0.0043 | 0.0024 | 0.0024 | NA |
| (Wilcoxon) | | | | | |
| P-value (JT) | NA | NA | NA | NA | 7.08E−7 |
| pERK Signal, % | | | | | |
| Inhibition | | | | | |
| at Day 1 | | | | | |
| 24 hours | | | | | |
| N | 5 | 5 | 6 | 6 | 8 |
| Mean (SD) | 1.0 (34.44) | 72.9 (5.75) | 90.2 (7.71) | 97.6 (7.33) | 7.3 (20.78) |
| Median | 4.4 | 71.5 | 89.9 | 99.5 | 8.5 |
| Q1, Q3 | 3.8, 20.4 | 67.7, 77.2 | 82.0, 97.4 | 97.4, 100.4 | −8.5, 21.5 |
| Min, Max | −56.7, 33.0 | 67.6, 80.4 | 81.8, 100.4 | 83.7, 105.2 | −24.1, 39.3 |
| P-value | 0.9417 | 0.0043 | 0.0024 | 0.0024 | NA |
| (Wilcoxon) | | | | | |
| P-value (JT) | NA | NA | NA | NA | 2.38E−8 |
| pERK Signal, % | | | | | |
| Inhibition | | | | | |
| at Day 10 | | | | | |
| Predose | | | | | |
| N | 5 | 5 | 1 | 5 | 8 |
| Mean (SD) | 24.4 (30.25) | 93.5 (4.24) | 97.1 | 98.6 (4.27) | 11.8 (32.65) |
| Median | 41.3 | 93.4 | 97.1 | 100.4 | 8.0 |
| Q1, Q3 | −1.5, 42.6 | 93.2, 96.0 | 97.1, 97.1 | 98.0, 101.0 | −1.9, 18.5 |
| Min, Max | −14.3, 54.0 | 86.8, 98.0 | 97.1, 97.1 | 91.4, 102.0 | −34.0, 79.3 |
| P-value | 0.6084 | 0.0043 | 0.1752 | 0.0043 | NA |
| (Wilcoxon) | | | | | |
| P-value (JT) | NA | NA | NA | NA | 9.58E−6 |
| pERK Signal, % | | | | | |
| Inhibition | | | | | |
| at Day 10 - | | | | | |
| 1 hour | | | | | |
| N | 5 | 5 | 1 | 6 | 8 |
| Mean (SD) | 62.2 (21.00) | 96.8 (1.33) | 99.4 | 99.5 (2.71) | 6.4 (20.49) |
| Median | 62.7 | 97.1 | 99.4 | 100.2 | 3.7 |
| Q1, Q3 | 48.2, 76.8 | 96.0, 97.7 | 99.4, 99.4 | 98.8, 101.0 | −4.2, 14.8 |
| Min, Max | 35.8, 87.7 | 94.9, 98.2 | 99.4, 99.4 | 94.5, 102.2 | −23.3, 45.6 |
| P-value | 0.0068 | 0.0043 | 0.1752 | 0.0024 | NA |
| (Wilcoxon) | | | | | |
| P-value (JT) | NA | NA | NA | NA | 8.79E−8 |
| pERK Signal, % | | | | | |
| Inhibition | | | | | |
| at Day 10 - | | | | | |
| 3 hours | | | | | |
| N | 5 | 5 | 1 | 6 | 7 |
| Mean (SD) | 85.4 (7.45) | 98.3 (2.05) | 99.6 | 100.5 (2.58) | 2.8 (20.23) |
| Median | 88.3 | 98.7 | 99.6 | 100.5 | −1.1 |
| Q1, Q3 | 80.2, 90.7 | 98.5, 98.8 | 99.6, 99.6 | 99.4, 102.3 | −7.3, 9.1 |
| Min, Max | 75.1, 92.6 | 94.9, 100.5 | 99.6, 99.6 | 96.3, 103.7 | −26.1, 40.4 |
| P-value | 0.0058 | 0.0058 | 0.1904 | 0.0034 | NA |
| (Wilcoxon) | | | | | |
| P-value (JT) | NA | NA | NA | NA | 2.19E−7 |
| pERK Signal, % | | | | | |
| Inhibition | | | | | |
| at Day 10 - | | | | | |
| 12 hours | | | | | |
| N | 5 | 5 | 1 | 6 | 7 |
| Mean (SD) | 43.5 (24.59) | 94.0 (4.12) | 96.7 | 100.2 (1.91) | −20.9 (18.34) |
| Median | 48.3 | 95.7 | 96.7 | 100.2 | −14.2 |
| Q1, Q3 | 18.3, 61.9 | 92.5, 95.8 | 96.7, 96.7 | 99.4, 101.4 | −31.6, −10.6 |
| Min, Max | 17.9, 71.2 | 87.5, 98.2 | 96.7, 96.7 | 97.1, 102.7 | −53.3, 3.9 |
| P-value | 0.0058 | 0.0058 | 0.1904 | 0.0034 | NA |
| (Wilcoxon) | | | | | |
| P-value (JT) | NA | NA | NA | NA | 2.99E−8 |
| pERK Signal, % | | | | | |
| Inhibition | | | | | |
| at Day 10 - | | | | | |
| 24 hours | | | | | |
| N | 5 | 5 | 6 | 5 | 8 |
| Mean (SD) | 12.6 (43.26) | 92.2 (3.83) | 98.3 (0.78) | 103.1 (2.58) | −11.5 (40.35) |
| Median | 39.8 | 91.6 | 98.5 | 101.5 | 3.9 |
| Q1, Q3 | −30.8, 43.6 | 90.0, 93.4 | 97.8, 98.8 | 101.2, 105.5 | −25.6, 13.8 |
| Min, Max | −38.2, 48.9 | 87.9, 98.0 | 97.1, 99.2 | 101.0, 106.2 | −98.0, 22.1 |
| P-value | 0.2723 | 0.0043 | 0.0024 | 0.0043 | NA |
| (Wilcoxon) | | | | | |
| P-value (JT) | NA | NA | NA | NA | 4.31E−9 |

P-value (Wilcoxon): Wilcoxon Rank Sum test comparing treatment arms to pooled placebo.
P-value (JT): Jonckheere's Trend Test indicating probability of dose-dependent response.

The results from both the SAD and MAD Cohorts demonstrate that administration of Compound 1 effected a dose-dependent and reversible inhibition of LPS-stimulated pERK in ex vivo whole blood samples, which is indicative of TPL2 pathway inhibition. Unexpectedly, each dose tested was effective at inhibiting the TPL-2 pathway.

The results further demonstrate that administration of 300 mg Compound 1 QD, 900 mg Compound 1 QD, and 600 mg Compound 1 BID each achieved a mean steady-state exposure to Compound A that resulted in >90% inhibition of LPS-stimulated pERK 24 hours post-last dose, also indicative of inhibition of TPL-2.

### Example 2. Single- and Multiple-Dose Study in Healthy Participants to Evaluate Drug-Drug Interactions and Food Effect

A Phase 1, open-label, single-center, multiple-cohort study was conducted to evaluate the effect of a mixed OATP/P-gp inhibitor (cyclosporine A; CsA) or food on the PK of Compound 1, the effect of Compound 1 on the PK of midazolam (MDZ), the effect of Compound 1 on the PK of OATP/CYP3A/BCRP substrates using atorvastatin (ATV), pravastatin (PRA), and rosuvastatin (ROS), and the effect of Compound 1 on the PK of a representative combined oral contraceptive (drospirenone/ethinyl estradiol; DRSP/EE), in healthy participants. 70 participants ages 18 to 55 years (inclusive) and having BMI of ≥18.0 kg/m² and ≤30.0 kg/m² were enrolled. For Cohorts 1-3, there were an approximately even distribution of healthy participants assigned male at birth and nonpregnant, nonlactating participants assigned female at birth. Cohort 4 included only healthy, nonpregnant, nonlactating participants assigned female at birth.

### Cohort 1—Mixed OATP/P-gp Inhibitor: CsA 600 mg

Participants in Cohort 1 were randomized to 1 of 2 treatment sequences (ABC or BAC), in a 1:1 ratio.

Treatment A: Single dose of Compound 1 600 mg, administered in the AM, under fasted conditions

Treatment B: Single dose of Compound 1 600 mg, administered in the AM, within 5 minutes of completing a high-fat/high-calorie meal

Treatment C: Single dose of Compound 1 600 mg, administered in the AM, under fasted conditions with a single dose of CsA 600 mg

TABLE 9

| | Study Days | | | | |
|---|---|---|---|---|---|
| Se- | Period 1 | | Period 2 | | Period 3 |
| quence | 1 | 2-5 | 1 | 2-5 | 1 |
| ABC | Cpd 1, 600 mg | WO | Cpd 1, 600 mg | WO | Cpd 1, 600 mg |
| | Single dose, | | Single dose + | | Single dose + |
| | Under fasted | | High-fat/high- | | CsA, 600 mg |
| | condition | | calorie meal | | Single dose, |
| BAC | Cpd 1, 600 mg | WO | Cpd 1, 600 mg | WO | Under fasted |
| | Single dose + | | Single dose, | | condition |
| | High-fat/high- | | Under fasted | | |
| | calorie meal | | condition | | |

CsA = cyclosporine A;
WO = washout

### Cohort 2—CYP3A Substrate: MDZ 2 mg

Participants in Cohort 2 received treatment sequence DEFG.

Treatment D: Single dose of MDZ 2 mg, administered under fasting condition on Day 1 of Period 1

Treatment E: Compound 1 600 mg, administered under fasting condition once daily, for 11 days on Days 1 to 11 of Period 2

Treatment F: Single dose of MDZ 2 mg, administered with a dose of Compound 1 600 mg, under fasting condition on Day 12 of Period 2

Treatment G: A dose of Compound 1 600 mg, administered under fasting condition on Day 13 of Period 2

TABLE 10
Study Days

| Period 1 | | Period 2 | | |
|---|---|---|---|---|
| 1 | | 1-11 | 12 | 13 |
| D: | 1-day | E: | F: | G: |
| MDZ, 2 mg | WO | Cpd 1, | MDZ, 2 mg, | Cpd 1, |
| Single dose, | | 600 mg QD, | Single dose + | 600 mg, |
| Under | | Under | Cpd 1, 600 mg, | Under |
| fasting | | fasting | Under | fasting |
| condition | | condition | fasting | condition |
| | | | condition | |

MDZ = midazolam;
QD = once daily;
WO = washout

Cohort 3—OATP/CYP3A Substrate: ATV 40 mg; or OATP/BCRP Substrate Cocktail: PRA 40 mg+ROS 10 mg

Participants in Cohort 3 received treatment sequence HIJKLMN.

Treatment H: Single dose of ATV 40 mg, administered under fasting condition on Day 1 of Period 1

Treatment I: Single doses of PRA+ROS 40 mg/10 mg, simultaneously administered under fasting condition on Day 1 of Period 2

Treatment J: Compound 1 600 mg, administered under fasting condition once daily on Days 1 to 5 of Period 3

Treatment K: Single dose of ATV 40 mg, administered with a dose of Compound 1 600 mg, under fasting condition on Day 6 of Period 3

Treatment L: Compound 1 600 mg, administered under fasting condition once daily on Days 7 to 9 of Period 3

Treatment M: A dose of Compound 1 600 mg, administered under fasting condition with single simultaneously administered doses of PRA+ROS 40 mg/10 mg on Day 10 of Period 3

Treatment N: Compound 1 600 mg, administered under fasting condition once daily on Days 11 to 13 of Period 3

TABLE 11
Study Days

| Period 1 | | Period 2 | | Period 3 | | | | |
|---|---|---|---|---|---|---|---|---|
| 1 | 2-4 | 1 | 2-4 | 1-5 | 6 | 7-9 | 10 | 11-13 |
| H: | WO | I: | WO | J: Cpd 1, | K: | L: | M: | N: |
| ATV, | | PRA + | | 600 mg | ATV, | Cpd 1, | PRA + | Cpd 1, |
| 40 mg | | ROS, | | QD, | 40 mg | 600 mg | ROS, | 600 mg |
| Single | | 40 mg/ | | Under | Single | QD, | 40 mg/ | QD, |
| dose, | | 10 mg | | fasting | dose + | Under | 10 mg | Under |
| Under | | Single | | condition | Cpd 1, | fasting | Single | fasting |
| fasting | | doses, | | | 600 mg | condition | doses + | condition |
| condition | | Under | | | dose, | | Cpd 1, | |
| | | fasting | | | Under | | 600 mg | |
| | | condition | | | fasting | | dose, | |
| | | | | | condition | | Under | |
| | | | | | | | fasting | |
| | | | | | | | condition | |

ATV = atorvastatin; PRA = pravastatin; QD = once daily; ROS = rosuvastatin; WO = washout

### Cohort 4—Representative Combined Oral Contraceptive: DRSP 3 mg and EE 0.02 mg

Participants in Cohort 4 received treatment sequence OPQR.

Treatment O: Single dose of DRSP 3 mg and EE 0.02 mg, administered under fasting condition on Day 1 of Period 1

Treatment P: Compound 1 600 mg, administered under fasting condition once daily on Days 1 to 11 of Period 2

Treatment Q: Single dose of DRSP 3 mg and EE 0.02 mg, administered with a dose of Compound 1 600 mg, under fasting condition on Day 12 of Period 2

Treatment R: Compound 1 600 mg, administered under fasting condition once daily on Days 13 to 15 of Period 2

TABLE 12
Study Days

| Period 1 | | Period 2 | | |
|---|---|---|---|---|
| 1 | 2-4 | 1-11 | 12 | 13-15 |
| O: | WO | P: | Q: | R: |
| DRSP/EE, | | Cpd 1, | DRSP/EE, | Cpd 1, |
| 3 mg/0.02 mg | | 600 mg QD, | 3 mg/0.02 mg | 600 mg QD, |
| Single dose, | | Under | Single dose + | Under |
| Under | | fasting | Cpd 1, 600 | fasting |
| fasting | | condition | mg dose, | condition |
| condition | | | Under | |
| | | | fasting | |
| | | | condition | |

DRSP = drospirenone;
EE = ethinyl estradiol;
QD = once daily;
WO = washout

Plasma PK sampling occurred relative to dosing of Compound 1 at the following time points for each cohort as specified below.

### Cohort 1:

Periods 1, 2, and 3: Day 1, 0 (pre-dose, ≤5 min before dose), 0.25, 0.5, 1, 1.5, 2, 3, 4, 6, 8, 12, 18, 24, 36, 48, 72, and 96 hours post-dose; and at the early termination (ET) visit, as applicable

### Cohort 2:

Period 1: Day 1, 0 (pre-dose, ≤5 min before dose), 0.25, 0.5, 1, 1.5, 2, 3, 4, 6, 8, 12, 18, and 24 hours post-dose; and at the ET visit, as applicable
Period 2: Day 1, 0 (pre-dose, ≤5 min before dose), 0.5, 1, 1.5, 2, 3, 4, 6, 8, 12, 18, and 24 hours post-dose; Day 12, 0 (pre-dose, ≤5 min before dose), 0.25, 0.5, 1, 1.5, 2, 3, 4, 6, 8, 12, 18, 24 hours post-dose; Day 13, 0 (pre-dose, ≤5 min before dose), 0.5, 1, 1.5, 2, 3, 4, 6, 8, 12, 18, 24, 48, 72 and 96 hours post-dose; and at the ET visit, as applicable

### Cohort 3:

Periods 1 and 2: Day 1, 0 (pre-dose, ≤5 min before dose), 0.5, 1, 1.5, 2, 3, 4, 6, 8, 12, 18, 24, 48, 72 and 96 hours post-dose, and at the ET visit, as applicable (The 96-hour post-dose PK samples for Day 1 of Periods 1 and 2 were collected pre-dose on Day 1 of Periods 2 and 3, respectively)
Period 3: Day 6, 0 (pre-dose, ≤5 min before dose), 0.5, 1, 1.5, 2, 3, 4, 6, 8, 12, 18, 24, 48, 72, and 96* hours post-dose; Day 10, 0 (pre-dose, ≤5 min before dose), 0.5, 1, 1.5, 2, 3, 4, 6, 8, 12, 18, 24, 48, 72 and 96* hours post-dose; and at the ET visit, as applicable (The Day 6, 96-hour post-dose sample was collected pre-dose on Day 10 of Period 3. The Day 10, 96-hour post-dose sample was collected on Day 14 of Period 3)

### Cohort 4:

Period 1: Day 1, 0 (pre-dose, ≤5 min before dose), 0.25, 0.5, 1, 1.5, 2, 3, 4, 6, 8, 12, 18, 24, 36, 48, 72, and 96* hours post-dose; and at the ET visit, as applicable (The 96-hour post-dose PK sample for Day 1 of Period 1 was collected pre-dose on Day 1 of Period 2)
Period 2: Day 12, 0 (pre-dose, ≤5 min before dose), 0.25, 0.5, 1, 1.5, 2, 3, 4, 6, 8, 12, 18, 24, 36, 48, 72, and 96 hours post-dose; and at the ET visit, as applicable

A time window of ±10% was allowed for PK samples collected through the 5 hours post-dose assessment. All other PK samples collected beyond 5 hours post-dose had a ±30-minute window.

Concentrations of Compound 1 and its active metabolites Compound A and Compound B, MDZ and its metabolite 1-OH-MDZ, ATV and its metabolite 2-OH-ATV, PRA, ROS, DRSP, and EE in plasma PK samples were determined by validated high performance liquid chromatography tandem mass spectrometry (LC-MS/MS) bioanalytical methods.

Safety assessments included monitoring of adverse events (AEs) and concomitant medications, clinical laboratory analyses, estimated glomerular filtration rate (eGFR) based on cystatin C, vital sign measurements, electrocardiograms (ECGs), and physical examinations.

### Pharmacokinetic Results

Pharmacokinetic parameters were determined for Compound 1 and its metabolites for all cohorts, MDZ and 1-OH-MDZ for Cohort 2; ATV, 2-OH-ATV, PRA, and ROS for Cohort 3; and DRSP and EE for Cohort 4. The parameters are set forth in Table 13, below.

TABLE 13

| Parameter | Definition |
|---|---|
| Cₘₐₓ | maximum observed concentration of drug |
| AUC_{inf} | area under the concentration versus time curve extrapolated |
| | to infinite time, calculated as AUCₗₐₛₜ + (Cₗₐₛₜ/λ_{z}) |
| AUCₗₐₛₜ | area under the concentration versus time curve from time |
| | zero to the last quantifiable concentration |
| Tₘₐₓ | time (observed time point) of Cₘₐₓ |
| t_{1/2} | terminal elimination half-life |

Table 14 shows Compound A plasma PK parameters and statistical comparisons after single-dose oral administration of Compound 1 600 mg under fasting and non-fasting (high-fat/high-calorie meal) conditions.

TABLE 14

| | Cpd 1 600 mg | Cpd 1 600 mg | |
|---|---|---|---|
| | Non-fasting | Fasting | |
| Cpd A (ng/mL) PK | (Test) | (Reference) | % GLSM |
| Parameter | Treatment: B | Treatment: A | Ratio |
| Mean (% CV) | (n = 14) | (n = 14) | (90% CI) |
| AUC_{inf} (h*ng/mL) | 86200 | 46300 | 192 |
| | (54.5) | (61.2) | (168, 221) |
| AUCₗₐₛₜ (h*ng/mL) | 72900 | 39900 | 187 |
| | (44.0) | (49.9) | (163, 215) |
| Cₘₐₓ (ng/mL) | 2280 | 1990 | 119 |
| | (24.6) | (38.2) | (104, 137) |
| Tₘₐₓ (h)^{a} | 5.00 | 4.00 | |
| | (4.00, 6.00) | (3.00, 4.00) | |
| t_{1/2} (h)^{a} | 32.6 | 29.8 | |
| | (23.2, 42.3) | (27.0, 33.0) | |

% CV = percentage coefficient of variation;
CI = confidence interval;
GLSM = geometric least-squares mean;
n = number of participants in the PK Analysis Set;
PK = pharmacokinetic;
Q1 = first quartile;
Q3 = third quartile
^{a}Values are presented as median (Q1, Q3)
Treatment A: Cpd 1 600 mg, fasted;
Treatment B: Cpd 1 600 mg, high-fat/high-calorie meal
Means presented are unadjusted arithmetic means.

Compared with a single-dose administration of Compound 1 600 mg under fasting conditions, a high-fat/high-calorie meal increased the AUC_{inf} of Compound A by 92% with no change in Cₘₐₓ. Food intake also slightly delayed absorption of Compound A: the median Tₘₐₓ increased from 4 hours while fasting to 5 hours with a high-fat/high-calorie meal.

Co-administration of Compound 1 with the mixed OATP/P-gp inhibitor CsA increased Compound A Cₘₐₓ and AUC_{inf} by 27% and 122%, respectively.

Co-administration of Compound 1 (600 mg) with the probe CYP3A substrate MDZ resulted in an increase in the Cₘₐₓ and AUC_{inf} of MDZ by 54% and 99%, respectively, relative to the administration of MDZ alone.

Co-administration of Compound 1 (600 mg) with the probe OATP/BCRP substrate and CYP3A substrate ATV resulted in an increase in the Cₘₐₓ and AUC_{inf} of ATV by 61% and 206%, respectively, relative to the administration of ATV alone.

Co-administration of Compound 1 (600 mg) with the probe OATP/BCRP substrate PRA (with ROS) resulted in an increase in the Cₘₐₓ and AUC_{inf} of PRA by 204% and 173%, respectively, relative to the administration of PRA+ROS alone.

Co-administration of Compound 1 (600 mg) with the probe OATP/BCRP substrate ROS (with PRA) resulted in an increase in the Cₘₐₓ and AUC_{inf} of ROS by 780% and 367%, respectively, relative to the administration of PRA+ROS alone.

Co-administration of Compound 1 (600 mg) with the representative combined oral contraceptive DRSP/EE resulted in a decrease in Cₘₐₓ of DRSP by 49% and no change in AUC_{inf} of DRSP and an increase in AUC_{inf} of EE by 75% with no change in Cₘₐₓ, relative to the administration of DRSP/EE alone.

### Example 3. Dose-Ranging Study in Participants with Moderately to Severely Active Ulcerative Colitis

A Phase 2, double-blinded, randomized, placebo-controlled, dose-ranging study in participants with moderately to severely active ulcerative colitis (UC) is conducted to evaluate the efficacy and safety of Compound 1. Approximately 176 participants assigned male at birth, or nonpregnant, nonlactating participants assigned female at birth, 18 to 75 years of age, with moderately to severely active UC are enrolled.

The study includes:
Screening Period: Days −30 to −1
Blinded Treatment Phase: Day 1 to Week 12

At Baseline/Randomization (Day 1), participants are randomized in a 1:1:1:1 ratio to 1 of 4 treatment groups as follows:
Treatment group 1 (n=44): Compound 1 600 mg once daily
Treatment group 2 (n=44): Compound 1 300 mg once daily
Treatment group 3 (n=44): Compound 1 150 mg once daily
Treatment group 4 (n=44): Placebo once daily

At Week 12 visit, participants have an efficacy assessment to determine if they achieved Clinical Response. Study drug assignments at Week 12 are based on Day 1 Blinded Treatment Phase assignments and Clinical Response status at Week 12. Participants who achieve Clinical Response at Week 12 continue the Blinded Treatment Phase. Participants who do not achieve Clinical Response at Week 12 are eligible for the Non-responder Treatment Phase.

Participants with insufficient data to assess Clinical Response at Week 12 discontinue study drug, are not eligible for the Non-responder Treatment Phase, and undergo the Blinded Treatment Phase Week 12 assessments followed by the posttreatment (PTx) assessments 30 days after the last dose of study drug.

### Blinded Treatment Phase: Week 12 to Week 52

Treatment assignments for participants who achieve Blinded Treatment Phase Week 12 are based on Blinded Treatment Phase Day 1 assignments:
Treatment group 1: Compound 1 300 mg once daily
Treatment group 2: Compound 1 300 mg once daily
Treatment group 3: Compound 1 150 mg once daily
Treatment group 4: Compound 1 150 mg once daily

### Non-Responder Treatment Phase: Day 1 to Week 12

Non-responder Treatment Phase Day 1 visit occurs on the same day as the Blinded Treatment Phase Week 12 visit.

In the Non-responder Treatment Phase, participants receive 600 mg or 300 mg of Compound 1 once daily in a double-blinded fashion up to Non-responder Treatment Phase Week 12. Treatment assignments for participants who did not achieve Clinical Response at Blinded Treatment Phase Week 12 are based on Blinded Treatment Phase Day 1 assignments:
Treatment group 1: Compound 1 600 mg once daily
Treatment group 2: Compound 1 300 mg once daily
Treatment group 3: Compound 1 300 mg once daily
Treatment group 4: Compound 1 600 mg once daily

### Non-Responder Treatment Phase: (Week 12 to Week 52)

At Non-responder Treatment Phase Week 12 visit, participants have an efficacy assessment to determine if they achieve Clinical Response using the locally scored Endoscopic Findings. Participants who achieve Clinical Response receive Compound 1 300 mg once daily up to Non-responder Treatment Phase Week 52.

Participants who do not achieve Clinical Response or with insufficient data to assess Clinical Response at Non-responder Treatment Phase Week 12 discontinue study drug and undergo the Non-responder Treatment Phase Week 12 assessments followed by the PTx assessments 30 days after the last dose of study drug.

### Week 12 Primary Analysis

The Week 12 Primary Analysis is conducted when all randomized participants complete Blinded Treatment Phase Week 12 visit or discontinue from study, and associated safety and efficacy assessments are completed.

After the Week 12 Primary Analysis, participants still in the Blinded Treatment Phase (between Week 12 and Week 52) may remain on the same dose as their assigned study drug regimen at Week 12. These participants may continue the Blinded Treatment Phase study visit schedule up to Week 52. Participants in the Non-responder Treatment Phase continue the Non-responder Treatment Phase study visit schedule up to Week 52. Participants choosing not to continue on study following the Week 12 Primary Analysis, attend the clinic at their next scheduled study visit or earlier, and undergo the ET assessments followed by the PTx assessments 30 days after the last dose of study drug.

### Posttreatment (PTx) Assessments

Participants who discontinue study drug return 30 days after the last dose for PTx assessments. Participants who discontinue study drug≥30 days prior to their ET visit will not be required to complete a separate posttreatment assessment.

Compound 1 is administered as tablets in strengths of 150 mg or 300 mg. In addition to the active ingredient, Compound 1 tablets also contain lactose monohydrate, microcrystalline cellulose, crospovidone, magnesium stearate, polyvinyl alcohol, titanium dioxide, polyethylene glycol, and talc.

PTM is administered as PTM-1 and PTM-2 tablets identical in size, shape, color, and appearance to corresponding active Compound 1 tablets. PTM-1 and PTM-2 tablets contain lactose monohydrate, microcrystalline cellulose, croscarmellose sodium, magnesium stearate, polyvinyl alcohol, titanium dioxide, polyethylene glycol, and talc.

Compound 1 is administered on an empty stomach; food can be consumed 4 hours before and 2 hours after the administration of Compound 1.

### Efficacy Assessments

The Mayo Clinic Score (MCS) is a scoring system for assessment of UC activity and utilizes 4 subscores: Stool Frequency, Rectal Bleeding, Endoscopic Findings, and the Physician Global Assessment (PGA). Each subscore is graded from 0 to 3 and the sum of the subscores (ranging from 0 to 12) determines the overall MCS. The modified MCS scoring system utilizes the Stool Frequency, Rectal Bleeding, and Endoscopic Findings subscores but excludes the PGA. The partial MCS scoring system utilizes the Stool Frequency, Rectal Bleeding, and PGA but excludes Endoscopic Findings subscores.

The Stool Frequency and Rectal Bleeding subscores are recorded in the participant's electronic diary (e-Diary) and are collected daily. The Endoscopic Finding subscore is reported by the central reader as well as the endoscopist performing the procedure (local reader) during Screening, Week 12, and Week 52.

Clinical Response is defined as a decrease from baseline of ≥2 points and at least 30% in 3 components of the modified Mayo Clinic Score (MCS)—Stool Frequency, Rectal Bleeding, and Endoscopic Findings—in addition to a ≥1 point decrease from baseline in the Rectal Bleeding subscore or Rectal Bleeding subscore of ≤1 at Week 12.

Clinical Remission is defined as a Stool Frequency subscore≤1 and not greater than baseline, Rectal Bleeding subscore of 0, and Endoscopic Findings subscore≤1 at Week 12.

Endoscopic Response is defined as an Endoscopic Findings subscore≤1 at Week 12.

Histologic Endoscopic Mucosal Improvement is defined as an Endoscopic Findings subscore≤1 and Geboes score≤3.1 (indicating neutrophil infiltration in <5% of crypts, no crypt destruction and no erosions, ulcerations, or granulation tissue) at Week 12.

### Safety Assessments

Safety assessments include monitoring of adverse events (AEs) and concomitant medications, clinical laboratory analyses, vital sign measurements, electrocardiograms (ECGs), and physical examinations.

### Pharmacokinetic Assessments

For all participants, blood samples for PK analysis are collected after study drug dosing at Week 2, 4, 8, 12, 24, 52 (at least 30 minutes and up to 3 hours after study drug dosing). Blood samples for PK analysis at Week 4 and Week 12 are collected prior to study drug administration.

Plasma concentrations of Compound 1 are analyzed. Additional analyses (eg, Compound 1 metabolites) are performed as applicable.

### Rationale for Dose Selection and Projected Outcomes

A population PK model for Compound 1 was developed and incorporated early clinical data, including 63 healthy volunteers' data from the Phase 1 clinical studies described in Examples 1 and 2, including single dose range of 300 to 1500 mg and multiple dose range of 75 to 900 mg. Nonlinear mixed effects modeling software (NONMEM) was used for the analysis. The pharmacokinetics of Compound A were adequately described by a two-compartment model featuring three transit absorption compartments and first-order elimination.

The highest dose (600 mg) of Compound 1 was selected to ensure maximum target coverage throughout the dosing interval in the majority of participants. It is projected that approximately 90% of study participants will achieve steady-state trough concentrations greater than the IC₉₀, based on the ex-vivo LPS-stimulated phosphorylated ERK (pERK) assay in human whole blood, following administration of Compound 1 600 mg once daily. Furthermore, approximately 60% and 20% of the participants are estimated to achieve greater than the IC₉₀ target coverage following the administration of Compound 1 300 mg once daily and 150 mg once daily, respectively. Table 15 below reflects the projected percentage of target (TPL2 kinase) coverage at the various doses tested.

TABLE 15

| | | Duration of time (h) |
|---|---|---|
| | Percentage (%) of | participants projected to |
| Compound 1 | participants projected to | achieve IC₉₀ or greater |
| Dose (mg) | achieve IC₉₀ or greater | Median (p5, p95) |
| 150 | 20 | 13 (0, 24) |
| 300 | 63 | 24 (9, 24) |
| 600 | 91 | 24 (22, 24) |

Across the dose range of Compound 1, the selected doses of 600 mg, 300 mg, and 150 mg are expected to have limited overlap in Compound A exposure based on the data across the Compound 1 dose range of 75 to 900 mg observed in the dose escalation study as described in Example 1. As such, the 150, 300 and 600 mg doses selected for the dose ranging study as described in Example 3 are expected to achieve the therapeutic exposures in patients with UC across all timepoints of the study (i.e., during the induction and maintenance phases) and to establish the plateau of therapeutic response.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter and is not intended to limit the appended claims to the specific embodiments illustrated.

1-10. (canceled)

11. A method of treating IBD in a patient in need thereof, comprising administering to the patient a prodrug which is: 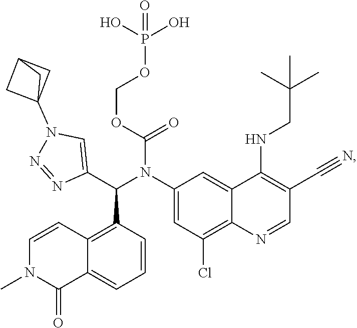 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]
or a pharmaceutically acceptable salt thereof,
wherein the prodrug is administered in a dosage of about 75 mg/dose to about 1,500 mg/dose.

12. (canceled)

13. The method of claim 11, wherein the prodrug is administered in a dosage of about 150 mg/dose to about 600 mg/dose.

14-17. (canceled)

18. The method of claim 11, wherein the prodrug is administered once daily.

19. (canceled)

20. The method of claim 11, wherein the prodrug is administered with food.

21-32. (canceled)

33. A method of treating IBD in a patient in need thereof, comprising administering to the patient a prodrug which is: 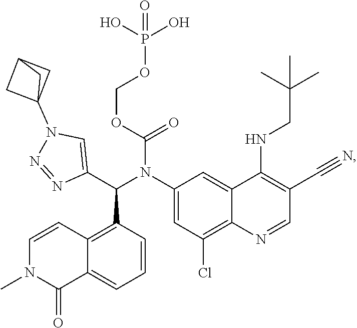 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]
or a pharmaceutically acceptable salt thereof,
wherein the prodrug is administered:
in a first dosage of about 150 mg/dose to about 1,500 mg/dose, for a first period of up to about 24 weeks; and then
in a second dosage less than or equal to the first dosage, for a second period.

34-53. (canceled)

54. A method of treating IBD in a patient in need thereof, comprising:
administering to the patient a prodrug which is: 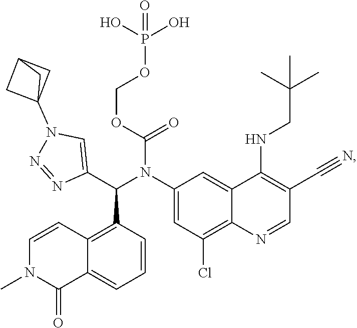 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]
in a first dosage of about 150 mg/dose, for a first period of up to about 24 weeks;
determining that the patient did not achieve an adequate response to the first dosage after the first period; and
administering the prodrug to the patient in a second dosage of about 300 mg/dose, for a second period.

55. The method of claim 33, wherein the first period is about 12 weeks.

56. The method of claim 33, wherein the second period is at least about 12 weeks.

57-72. (canceled)

73. A pharmaceutical composition comprising a prodrug which is: 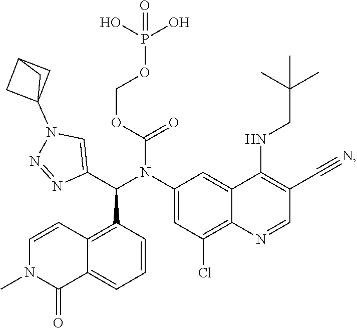 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]
wherein the pharmaceutical composition further comprises a diluent, a disintegrant, or a lubricant, or any combination thereof.

74. The pharmaceutical composition of claim 73, wherein the pharmaceutical composition comprises lactose monohydrate, microcrystalline cellulose, crospovidone, or magnesium stearate, or any combination thereof.

75-78. (canceled)

79. The pharmaceutical composition of claim 73 in a dosage form, wherein the dosage form is a tablet.

80. The pharmaceutical composition of claim 79, wherein the tablet comprises a film coating.

81-82. (canceled)

83. The pharmaceutical composition of claim 80, wherein the film coating comprises polyvinyl alcohol, polyethylene glycol, or talc, or any combination thereof.

84. An oral dosage form comprising a prodrug of Compound A: 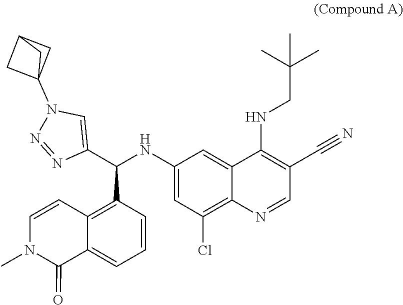 [SMILES: Cn1ccc2c([C@H](Nc3cc(Cl)c4ncc(C#N)c(NCC(C)(C)C)c4c3)c3cn(C45CC(C4)C5)nn3)cccc2c1=O]
or a pharmaceutically acceptable salt of the prodrug, and a pharmaceutically acceptable excipient,
wherein the oral dosage form is formulated to achieve one or more pharmacokinetic characteristics selected from:
an arithmetic mean steady-state AUCₜₐᵤ of Compound A of about 10 h*μg/mL to about 80 h*μg/mL;
an arithmetic mean steady-state Cₘₐₓ of Compound A of about 0.9 μg/mL to about 9 μg/mL;
a median steady-state Tₘₐₓ of Compound A of about 1.5 hours to about 6 hours; and
a median steady-state t_{1/2} of Compound A of about 18 hours to about 30 hours,
upon daily oral administration of the dosage form to a human patient in a fasted state.

85. An oral dosage form comprising a prodrug of Compound A: 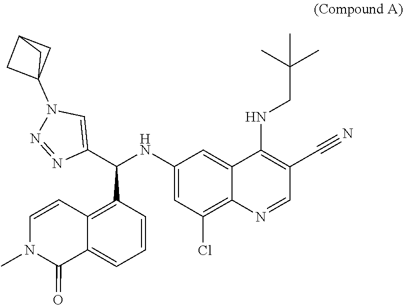 [SMILES: Cn1ccc2c([C@H](Nc3cc(Cl)c4ncc(C#N)c(NCC(C)(C)C)c4c3)c3cn(C45CC(C4)C5)nn3)cccc2c1=O]
and a pharmaceutically acceptable excipient,
wherein the oral dosage form is formulated to achieve one or more pharmacokinetic characteristics selected from:
an arithmetic mean steady-state AUCₜₐᵤ of Compound A of about 10 h*μg/mL to about 80 h*μg/mL;
an arithmetic mean steady-state Cₘₐₓ of Compound A of about 0.9 μg/mL to about 9 μg/mL;
a median steady-state Tₘₐₓ of Compound A of about 1.5 hours to about 6 hours; and
a median steady-state t_{1/2} of Compound A of about 18 hours to about 30 hours,
upon daily oral administration of the dosage form to a human patient in a fasted state,
wherein the prodrug is

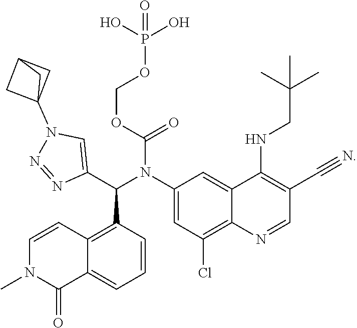 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

86-87. (canceled)

88. The oral dosage form of claim 85, formulated to achieve an arithmetic mean AUCₜₐᵤ of Compound A of about 10 h*μg/mL to about 65 h*μg/mL.

89-100. (canceled)

101. The method of claim 54, wherein the first period is about 12 weeks.

102. The method of claim 54, wherein the second period is at least about 12 weeks.

103. A method of treating IBD in a patient in need thereof, comprising administering to the patient a pharmaceutical composition comprising a prodrug which is: 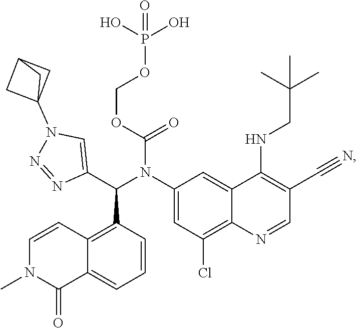 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]
wherein the pharmaceutical composition further comprises a diluent, a disintegrant, or a lubricant, or any combination thereof.
